(19) United States
(12) Patent Application Publication
(10) Pub. No.: US 2022/0259536 A1
(43) Pub. Date: Aug. 18, 2022

(54) MICROCHANNEL CELL CULTURE DEVICE AND SYSTEM

(71) Applicant: THE CHARLES STARK DRAPER LABORATORY, INC., Cambridge, MA (US)

(72) Inventors: Samuel Kann, Cambridge, MA (US); Joseph L. Charest, Cambridge, MA (US); Else M. Vedula, Stoneham, MA (US); Erin M. Shaughnessey, Somerville, MA (US); Hesham Azizgolshani, Belmont, MA (US); Brett Isenberg, Cambridge, MA (US); Jonathan R. Coppeta, Windham, NH (US)

(21) Appl. No.: 17/650,765

(22) Filed: Feb. 11, 2022

Related U.S. Application Data
(60) Provisional application No. 63/149,057, filed on Feb. 12, 2021.

(51) Int. Cl. C12M 1/32; C12M 3/06; C12M 1/00; C12M 1/34

(52) U.S. Cl. CPC C12M 23/12; C12M 23/16; C12M 23/34; C12M 41/32; C12M 41/34; C12M 41/12; C12M 41/26; C12M 23/22

(57) ABSTRACT

A microchannel cell culture device is disclosed. The microchannel cell culture device includes a well plate defining an array of tissue modeling environments. A cell culture system including the microchannel cell culture device is also disclosed. The cell culture system includes a plurality of optical sensors, a platform, and a light source. A method of high throughput screening cell biological activity with the microchannel cell culture device is disclosed. A method of measuring oxygen consumption rate of cells in the microchannel cell culture device is disclosed. A method of facilitating drug development with the microchannel cell culture device is also disclosed.

### CROSS REFERENCE TO RELATED APPLICATIONS

This application claims priority under 35 U.S.C. § 119(e) to U.S. Provisional Patent Application Ser. No. 63/149,057, titled “MICROCHANNEL CELL CULTURE DEVICE AND SYSTEM,” filed Feb. 12, 2021, which is incorporated by reference herein in its entirety for all purposes.

### FIELD OF TECHNOLOGY

Aspects and embodiments disclosed herein are generally related to microfluidic cell culture and more specifically, to systems and methods for monitoring cells in microfluidic cell culture.

### SUMMARY

In accordance with one aspect, there is provided a cell culture system. The cell culture system may comprise a microchannel cell culture device. The microchannel cell culture device may comprise a well plate formed of a plurality of structural layers and a membrane layer. The membrane layer may be positioned between two structural layers. The well plate may be defining an array of tissue modeling environments. Each tissue modeling environment may include a first fluid reservoir, a second fluid reservoir, a third fluid reservoir, and a fourth fluid reservoir. Each tissue modeling environment may include a first microchannel fluidically coupling the first fluid reservoir to the second fluid reservoir. Each fluid reservoir may include a second microchannel fluidically coupling the third fluid reservoir to the fourth fluid reservoir. At least a portion of the first microchannel may overlap at least a portion of the second microchannel with the membrane layer extending between the overlapping portions of the first and second microchannel. Each of the overlapping portions of the first and second microchannels may be optically transparent. The cell culture system may comprise a plurality of optical sensors. Each optical sensor may be positioned to scan a corresponding overlapping portion of the first and second microchannels on a bottom surface of the well plate. The cell culture system may comprise a platform configured to support a bottom surface of the microchannel cell culture device.

In some embodiments, the cell culture system may comprise a light source configured to be positioned adjacent the platform opposite the microchannel cell culture device. The light source may be configured to direct light towards the plurality of optical sensors. The cell culture system may comprise a meter operably connected to the light source.

In some embodiments, the optical sensors are formed of a nanoparticle solution.

In some embodiments, the light source is a fiber optic cable.

In some embodiments, the platform is movable.

In some embodiments, the optical sensor is configured to measure at least one of oxygen concentration, pH, temperature, and glucose concentration.

In some embodiments, the well plate comprises a light shielding layer positioned on a top surface of the well plate.

In some embodiments, each optical sensor has a length extending from the first fluid reservoir to the second fluid reservoir, and from the third fluid reservoir to the fourth fluid reservoir.

In some embodiments, each fluid reservoir may be configured to hold a column of fluid.

In accordance with another aspect, there is provided a microchannel cell culture device. The microchannel cell culture device may comprise a well plate defining an array of tissue modeling environments. Each tissue modeling environment may include at least one microchannel fluidically coupling a first fluid reservoir to a second fluid reservoir. A bottom surface of the microchannel may be optically transparent. The microchannel cell culture device may comprise a light shielding layer positioned adjacent a top surface of the at least one microchannel. The microchannel cell culture device may comprise a plurality of optical sensors. Each optical sensor may be positioned to scan the bottom surface of the at least one microchannel of a corresponding tissue modeling environment.

In some embodiments, each tissue modeling environment may include at least two microchannels, a first microchannel fluidically coupling the first fluid reservoir to the second fluid reservoir and a second microchannel fluidically coupling a third fluid reservoir to a fourth fluid reservoir.

In some embodiments, at least a portion of the first microchannel overlaps at least a portion of the second microchannel.

In some embodiments, the microchannel cell culture device further comprising a membrane layer extending between the overlapping portions of the first and second microchannels.

In some embodiments, a bottom surface of the overlapping portions of the first and second microchannels is optically transparent.

In accordance with another aspect, there is provided a method of high throughput screening cell biological activity. The method may comprise seeding at least one cell type onto at least one tissue modeling environment of a microchannel cell culture device. The microchannel cell culture device may comprise a well plate formed of a plurality of structural layers and a membrane layer, the membrane layer positioned between two structural layers and the well plate defining an array of the tissue modeling environments, each tissue modeling environment including a first fluid reservoir and a second fluid reservoir, and a microchannel fluidically coupling the first fluid reservoir to the second fluid reservoir a bottom surface of the microchannel being optically transparent, and a plurality of optical sensors, each optical sensor positioned to scan the bottom surface of the microchannel of a corresponding tissue modeling environment. The method may comprise introducing a pre-determined dose of at least one biologically active agent into the at least one tissue modeling environment. The method may comprise measuring a parameter within the at least one tissue modeling environment to produce a first measurement.

In some embodiments, the method may comprise positioning the microchannel cell culture device on a platform configured to support the bottom surface of the microchannel cell culture device. The method may comprise activating a light source positioned adjacent the platform opposite the microchannel cell culture device to direct light towards the plurality of optical sensors.

In some embodiments, the method may comprise after a pre-determined amount of time, measuring the parameter within the at least one tissue modeling environment to produce a second measurement. The method may comprise calculating a rate of change of the parameter from the first and second measurement to determine the cell biological activity of each cell type responsive to the at least one biologically active agent.

In some embodiments, the method may further comprise coupling the light source to a surface of the platform opposite the microchannel cell culture device.

In some embodiments, the parameter may be selected from oxygen concentration, pH, temperature, and glucose concentration.

In accordance with another aspect, there is provided a method of measuring oxygen consumption rate of cells. The method may comprise seeding the cells onto at least one tissue modeling environment of a microchannel cell culture device The method may comprise introducing an oxygen rich fluid into the at least one seeded tissue modeling environment. The method may comprise measuring a first oxygen concentration within the at least one seeded tissue modeling environment with the plurality of optical sensors. The method may comprise reducing flow rate of the oxygen rich fluid to induce a static environment within the at least one seeded tissue modeling environment. The method may comprise, after a pre-determined amount of time, measuring a second oxygen concentration within the at least one seeded tissue modeling environment with the plurality of optical sensors to determine the oxygen consumption rate of the cells.

In accordance with yet another aspect, there is provided a method of facilitating drug development. The method may comprise providing a cell culture system. The method may comprise providing instructions to seed at least one cell type onto at least one tissue modeling environment. The method may comprise providing instructions to measure a parameter within the at least one tissue modeling environment to produce a first measurement.

In some embodiments, the method may comprise providing instructions to activate a light source to direct light towards the plurality of optical sensors.

In some embodiments, the method may comprise providing instructions to after a pre-determined amount of time, measure the parameter within the at least one tissue modeling environment to produce a second measurement. The method may comprise providing instructions to calculate a rate of change of the parameter from the first and second measurement to determine the cell biological activity of the at least one cell type responsive to the at least one biologically active agent.

In some embodiments, the method may comprise providing a platform configured to support the bottom surface of the microchannel cell culture device.

In some embodiments, the method may comprise providing a light source configured to be positioned adjacent the platform opposite the microchannel cell culture device, the light source configured to direct light towards the plurality of optical sensors.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates an example apparatus for providing an array of tissue modeling environments, according to one embodiment;

FIG. 2 illustrates a magnified view of a tissue modeling environment provided by the example apparatus illustrated in FIG. 1;

FIG. 3A illustrates a top down view of the tissue modeling environment illustrated in FIG. 2;

FIG. 3B illustrates a perspective view of the tissue modeling environment illustrated in FIG. 2;

FIG. 3C illustrates a cross sectional view of the tissue modeling environment illustrated in FIG. 2;

FIG. 4A illustrates a top down view of two adjacent tissue modeling environments of the cell culture platform 105 in FIG. 1;

FIG. 4B illustrates the pump assembly interacting with the first and second tissue modeling environments shown in FIG. 4A;

FIG. 5A illustrates an exploded view of the cell culture platform of the example apparatus illustrated in FIG. 1;

FIG. 5B illustrates fluid pathways through the structural layers of the example cell culture platform illustrated in FIG. 5A;

FIG. 6A illustrates an exploded view of an example cell culture platform having an array of tissue modeling environments with integrated sensors, according to one embodiment;

FIG. 6B illustrates a top down view of the second structural layer of the example cell culture platform illustrated in FIG. 6A;

FIG. 6C illustrates an exploded view of an example cell culture platform having a single tissue modeling environment with integrated sensors, according to one embodiment;

FIG. 6D illustrates a cross sectional view the example cell culture platform shown in FIG. 6C;

FIG. 7A illustrates a cross section of a microchannel structure fabricated by embossing a plastic material, according to one embodiment;

FIG. 7B illustrates a cross section of a microchannel structure fabricated using a thru cut technique, according to one embodiment;

FIG. 8 illustrates an exploded view of an example cell culture platform fabricated using a thru cut technique, as previously shown in FIG. 7B;

FIGS. 9A-9F illustrates top down views of various example implementations of microchannel structures;

FIG. 10 illustrates views of an example pneumatic manifold actuator and pump assembly;

FIG. 11 illustrates a flow chart of an example method for populating cells into the cell culture platform of FIG. 1;

FIG. 12 illustrates a flow chart of an example experimental method for simulating hypoxic conditions in healthy tissue using the cell culture platform of FIG. 1;

FIG. 13 is a schematic drawing of a microchannel cell culture device and system, according to one embodiment; and

FIG. 14 includes a graph of air saturation over time and an image showing placement of an optical sensor in a microchannel, according to one embodiment;

FIG. 15A is a graph of oxygen pressure over time showing oxygen concentration, according to one embodiment; and

FIG. 15B is a graph of oxygen consumption rate over varying drug concentrations, according to one embodiment.

### DETAILED DESCRIPTION

Biological assays may be used to measure efficacy and potency of a biologically active agent by its effect on living cells or tissues. High throughput assays enable the performance of a large number of biological assays simultaneously. Biological agents, combinations thereof, and dosing schemes can be varied in a single test run against one or more cell and/or tissue samples to rapidly and efficiently obtain comprehensive results. Microfluidic cell culture systems may further increase capabilities of high throughput analysis by streamlining and automating experimental steps. There exists a need for integrated and efficient sensors on microfluidic cell culture devices to improve high throughput microfluidic data collection for biological assays. The systems and methods disclosed herein involve culturing cells within a microfluidic device having integrated optical sensors. The devices and systems may enable a label-free and contactless method for tissue metabolic monitoring in microfluidic cell culture systems. The devices and systems may avoid the need for invasive, slow, metabolic assays performed with conventional systems. In certain embodiments, existing microfluidic cell culture systems may be retrofit for performance of the methods disclosed herein. Furthermore, the devices may be compatible with existing biology lab equipment and workflow (such as microscopes, incubators). The devices and systems disclosed herein may improve high throughput biological assays of microfluidic systems by enabling metabolic monitoring of cells within microchannels.

In accordance with one aspect, there is provided a microchannel cell culture device. The microchannel cell culture device may comprise a well plate defining an array of tissue modeling environments. For example, the well plate may include 2, 4, 6, 12, 24, 48, 60, 72, 96, 384, or 1536 tissue modeling environments. Each tissue modeling environment may include a microchannel fluidically coupling a first fluid reservoir to a second fluid reservoir. Each fluid reservoir may be configured to hold a column of fluid. A bottom surface of the microchannel may be optically transparent.

As disclosed herein, optical transparency may refer to a material that allows a target percentage of optical light to pass through, for example, at least 75%, at least 80%, at least 85%, at least 90%, at least 95%, at least 98%, at least 99%, at least 99.9%, or at least 99.99% of optical light. The optical transparency may refer to a specific or selected wavelength. For example, the devices and components disclosed herein may be optically transparent to one or more of gamma-ray, x-ray, ultraviolet, visible, near-infrared, infrared, microwave, and radio wave light, as selected.

The microchannel cell culture device may be formed of a material that is substantially impermeable to oxygen. For example, the device may be formed of a low permeability polymer. The small volume of the microchannel allows for subtle changes in dissolved oxygen to be measured by the sensor due to cell oxygen consumption. Oxygen transfer from the atmosphere through the walls into the microfluidic channels is expected to be negligible because the microchannel materials have low permeability to oxygen.

The microchannels may be dimensioned to hold a small volume of liquid. For example, the microchannels may have a volume of less than 5 μL, for example, 1 μL to 4 μL, or 1 μL to 2 μL. Exemplary microchannels may have a width or diameter of 0.5 mm to 1 mm, for example, 0.7 mm to 1 mm. Exemplary microchannels may have a length of 5 mm to 10 mm, for example 6 mm to 8 mm between reservoirs. The microchannels may have a height of between about 200 μm to 300 μm, for example, about 250 μm.

The fluid reservoirs positioned at distal ends of the microchannel may each have a volume greater than the microchannel. The microchannel and reservoirs may be dimensioned to limit, inhibit, or reduce transfer of dissolved oxygen from a reservoir to the microchannel. By making diffusion of oxygen into the microchannel low or negligible, changes in oxygen concentration within the channel may be attributed to cell biological activity. Additionally, even in static conditions, the fluid within the reservoirs may remain oxygenated because of the significantly larger surface area interacting with air and significantly larger liquid volume and/or because cell oxygen consumption may be significantly less in the reservoir than in the microchannel.

The microchannel cell culture device may comprise a plurality of optical sensors. Each optical sensor may be positioned adjacent the bottom surface of a corresponding microchannel. A gap may be maintained between the bottom surface of the microchannel and the optical sensor. The gap may be sufficient to keep cells from contacting the optical sensor. In some embodiments, the gap may be, for example, between 100 μm to 300 μm, for example, between 150 μm to 250 μm. The optical sensor may be substantially centrally located adjacent the bottom surface of the microchannel. The optical sensor may be positioned at one end of the microchannel. The optical sensor may extend the length of the microchannel. More than one optical sensor may be positioned per microchannel. For example, optical sensors may be positioned at both ends of the microchannel. The optical sensor may be configured to measure a parameter within the microchannel. The optical sensor may be configured to measure biological activity of the cells within the tissue environment. For example, the optical sensor may be configured to measure metabolic activity of the cells.

In some embodiments, the optical sensor may be configured to measure concentration of an analyte. The analyte may include, for example, an analyte typically consumed by biologically active cells. Examples include oxygen and glucose. The analyte may include, for example, an analyte typically produced or secreted by biologically active cells. Examples include carbon dioxide, secretion factors, and transport ions. The analyte may include, for example, analytes having transport controlled by ion selective membrane technology. Other analytes are within the scope of the disclosure. The optical sensor may be configured to measure other biologically significant changes, such as pH and/or temperature. The optical sensor may be configured to measure other biologically significant parameters, such as pressure and/or relative humidity. In some embodiments, the bottom surface of the microchannel may be semi-permeable.

For example, the bottom surface may be permeable to the analyte being detected. The bottom surface may be permeable to a fluid within the microchannel. In general, the semi-permeable surface may be impermeable to cells. In one exemplary embodiment, the optical sensor may be an oxygen sensor. The bottom surface of the microchannel may be formed of an oxygen permeable membrane or scaffold. The semi-permeable surface may position the cells suspended above the sensor location, avoiding cell contact with the sensor probe.

In some embodiments, each fluid reservoir may include a second microchannel fluidically coupling third and fourth fluid reservoirs. At least a portion of the first microchannel may overlap at least a portion of the second microchannel. The overlapping portion of the microchannels may be optically transparent. The optical sensor may be positioned adjacent the overlapping portion of the microchannels, on a bottom surface. For example, the cells may be seeded in the upper microchannel, and the optical sensor may be positioned in the lower microchannel. The optical sensor may be substantially centrally located on the overlapping portion. The optical sensor may be positioned at one end of the overlapping portion. The optical sensor may extend the length of the overlapping portion. More than one optical sensor may be positioned in discreet locations along the overlapping portion. For example, optical sensors may be positioned at both ends of the overlapping portion.

A structural member may be positioned between the overlapping portions of the microchannels. The structural member may be a membrane, scaffold, filter, mesh, or other structural member. The structural member may allow diffusion of the analyte being measured, for example, oxygen. The structural member may allow diffusion of the fluid. In general, the structural member may be impermeable to cells. The structural member may function to mechanically support and position the cells within the upper microchannel to avoid contact between the cells and the deposited optical sensor in the lower microchannel.

The optical sensor may be dimensioned smaller than the length of the microchannel. The optical sensor may be positioned substantially centrally along the length of the microchannel, for example, between the first reservoir and the second reservoir. In some embodiments, the optical sensor may be positioned substantially centrally along the length of the overlapping portion of the first and second microchannels. In other embodiments, the optical sensor may be dimensioned larger or substantially equally to than the length of the microchannel. For example, the optical sensor may extend the length of the microchannel. The optical sensor may have a length extending from the first fluid reservoir to the second fluid reservoir. In certain embodiments, the optical sensor may also extend from the third fluid reservoir to the fourth fluid reservoir. In some embodiments, multiple optical sensors may be positioned in discreet locations along the length of the microchannel or the overlapping portion.

The optical sensor may be formed of a sensor spot. The sensor spot may be positioned on the bottom surface of the microchannel. The sensor spot may be contactless, for example, substantially free of leads or traces. The sensor spot may be suitable for measuring percent O₂ in gas or dissolved oxygen in liquid. An exemplary oxygen sensor spot is OXSP5 oxygen sensor spot, distributed by PyroScience GmbH, Aachen, Nordrhein-Westfalen, Germany. The sensor spot may be suitable for measuring pH. Exemplary pH sensor spots include PHSP5-PK5, PHSP5-PK6, PHSP5-PK7, PHSP5-PK8, and PHSP5-PK8T pH sensor spots distributed by PyroScience GmbH, Aachen, Nordrhein-Westfalen, Germany. The sensor spot may be suitable for measuring temperature. An exemplary temperature sensor spot is TPSP5 temperature sensor spot distributed by PyroScience GmbH, Aachen, Nordrhein-Westfalen, Germany. Other optical sensors may be used.

The optical sensor may be formed of a nanoparticle solution. For example, the optical sensor may be formed of a dispersible oxygen nanoparticle suitable for measuring dissolved oxygen. The nanoparticle solution may be painted onto the bottom surface of the microchannel. Thus, the size of the optical sensor formed of a nanoparticle solution variable. An exemplary nanoparticle optical sensor is Oxnano oxygen nanoprobes, distributed by PyroScience GmbH, Aachen, Nordrhein-Westfalen, Germany.

The oxygen sensors may be suitable for a measuring range of 0 to 500 hPa oxygen (0-250% air saturation). The pH sensors may be suitable for a measuring range of 4.0-6.0, 5.0-7.0, 6.0-8.0, or 7.0-9.0 pH units. The temperature sensors may be suitable for measuring 0° C.-50° C. The optical sensors may have a shelf life of at least about 3 years, with negligible drift. Other optical sensors may be used.

The microchannel cell culture device may comprise a light shielding layer. The light shielding layer may be positioned adjacent a top surface of the microchannel. In some embodiments, the light shielding layer may be positioned adjacent the overlapping portion of the microchannels, on a top surface. The light shielding layer may reduce glare. The light shielding layer may reduce intensity. The light shielding layer may be configured to substantially focus the light to a target portion of the microchannel cell culture device, for example to a target tissue modeling environment. The light shielding layer may be substantially opaque.

As disclosed herein, opacity and/or substantial opacity may refer to a material that blocks a target percentage of light from passing through, for example, at least 75%, at least 80%, at least 85%, at least 90%, at least 95%, at least 98%, at least 99%, at least 99.9%, or at least 99.99% of light. Opacity may refer to a specific or selected wavelength. For example, the devices and components disclosed herein may be substantially opaque to one or more of gamma-ray, x-ray, ultraviolet, visible, near-infrared, infrared, microwave, and radio wave light, as selected. The microchannel cell culture device disclosed herein may comprise any one or more feature of the bi-layer multi-well cell culture platform as disclosed in U.S. Patent Application Publication No. 2018/0142196 titled “Bi-layer multi-well cell culture platform” filed Nov. 21, 2017, herein incorporated by reference in its entirety for all purposes. Methods of retrofitting a microchannel cell culture device are disclosed herein. The methods may comprise providing one or more of an optical sensor, a platform, a light source, a meter, and a controller as disclosed herein. The methods may comprise providing instructions to install one or more of the optical sensor, the platform, the light source, the meter, and the controller onto a microchannel cell culture device, as disclosed herein.

In accordance with another aspect, there is provided a cell culture system. The cell culture system may comprise a microchannel cell culture device, as previously described. The microchannel cell culture device may be connectable to a source of cells for seeding each tissue modeling environment. The cell culture device may be connectable to a source of cell culture media. In certain embodiments, the cell culture device may be connectable to a source of an air saturated fluid. the air saturated fluid may be air saturated cell culture media. The air saturated fluid may be between 18%-21% air saturated. In certain embodiments, the air saturated fluid may be between 20%-21% air saturated. The air saturated fluid may be about 21% air saturated.

The system may comprise a pump configured to direct one or more fluid into and/or through the microchannel cell culture device. The pump may be configured to direct the fluid at a flow rate of about 0-200 μL/min, for example, about 0-100 μL/min or about 0-70 μL/min. In some embodiments, the pump may be operatively connected to a controller. The controller may be configured to instruct the pump to introduce fluid, for example, cell culture media, air saturated fluid, into the microchannel cell culture device in accordance with the methods disclosed herein.

The cell culture system may comprise a platform configured to support the bottom surface of the microchannel cell culture device. In some embodiments, the platform may be movable. For example, the platform may be movable on an x-y horizontal plane. In certain embodiments, the movement of the platform may be programmable. For example, the platform may be programmed to serially align each tissue modeling environment with a light source positioned opposite the microchannel cell culture device.

In some embodiments, the cell culture system may comprise a microscope. The platform may be a stage of the microscope. Thus, the stage may be configured to support the bottom surface of the microchannel cell culture device. The microscope may be positioned to image the cells within the tissue modeling environment.

The cell culture system may comprise a light source configured to be positioned adjacent the platform. The light source may be positioned adjacent the platform, opposite the microchannel cell culture device. The light source may be configured to direct light towards the plurality of optical sensors. Thus, the light source may be configured to direct light towards the bottom surface of the microchannel cell culture device. The light source may be configured to collect and transmit data from an optical sensor.

In some embodiments, the light source may be a fiber optic cable. The fiber optic cable may have a tip dimensioned to direct light to a single optical sensor. The fiber optic cable may have a tip diameter of 50 μm to 3 mm. For example, the fiber optic cable may have a tip diameter of 50 μm-70 μm, 230 μm, 430 μm, 1.5 mm, or 3.0 mm. The fiber optic cable may have a tapered tip. For example, the fiber optic cable may have a body diameter greater than the tip diameter. In exemplary embodiments, the fiber optic cable may have a body diameter of 3 mm and a tip diameter of 1.5 mm, a body diameter of 430 μm and a tip diameter of 230 μm, or a body diameter of 230 μm and a tip diameter of 50 μm-70 μm. Other light sources are within the scope of the disclosure.

In some embodiments, the system may comprise a plurality of light sources. The plurality of light sources may be positioned to correspond with a plurality of tissue modeling environments. For instance, the plurality of light sources may be positioned to direct light to a plurality of optical sensors substantially simultaneously. The plurality of light sources may be configured to obtain data from a plurality of optical sensors substantially simultaneously.

The cell culture system may comprise a meter operably connected to the light source. The meter may be configured to receive and process data from the light source. The meter may be configured to transmit data to a user interface. In some embodiments, the user interface is integrated into a single device with the meter. In other embodiments, the user interface is external to the meter. Exemplary meters include FireSting-O₂ and FireSting-PRO meters distributed by PyroScience GmbH, Aachen, Nordrhein-Westfalen, Germany.

The meter may be coupled to a controller or computing device, for example, a personal computer or mobile device, by analog, digital, or wireless connection. The controller or computing device may be programmed to receive and process data from the meter. The controller or computing device may calibrate the sensors, log data, display data for the user, and/or provide an interface for the user to operate the device or system. The controller or computing device may be programmed to process the data for calculating drift, rate of change of the parameter, or other features. The controller or computing device may be configured to alert a user responsive to the processed data.

The devices and systems disclosed herein may be used to measure biological activity of the cells. For example, the devices and systems disclosed herein may be used to measure metabolic activity of the cells. In some embodiments, the devices and systems disclosed herein may be used to perform biological assays. In particular, the devices and systems disclosed herein may be used to perform high throughput biological assays. In accordance with one embodiment, there is provided a method of screening cell biological activity in a high throughput assay. The method may comprise seeding at least one cell type onto a tissue modeling environment of the microchannel cell culture device. The cell type may be seeded onto a plurality of tissue modeling environments, for example, 6, 12, 24, 48, 96, or 384 tissue modeling environments. The method may comprise perfusing the cells with cell culture media. The method may comprise incubating the cells for a predetermined period of time. The method may comprise imaging the seeded cells. For example, the method may comprise imaging the seeded cells to confirm viability of the cells.

The method may comprise introducing a pre-determined dose of at least one biologically active agent into each tissue modeling environment. The biologically active agent may comprise one or more compound of interest. The assay may be performed by varying the dosage of the biologically active agent in across tissue modeling environments. The assay may be performed by varying the compound or combination of interest in across tissue modeling environments. The assay may be performed by varying cell type across tissue modeling environments.

In exemplary embodiments, the biological assay may be a drug development assay. For instance, the biological assay may be a toxicology assay. The methods disclosed herein may facilitate drug development. Drug development may be facilitated by providing a cell culture system as disclosed herein or any one or more component thereof. Drug development may be facilitated by providing instructions to measure cell biological activity as disclosed herein. In exemplary embodiments, drug development may be facilitated by providing instructions to measure oxygen consumption rate as disclosed herein.

The methods of performing a biological assay may comprise taking a first measurement of at least one parameter of the cells within each tissue modeling environment. The first measurement may be taken before dosing the cells with the biologically active agent. The first measurement may be taken substantially simultaneously while dosing the cells with the biologically active agent. The first measurement may be taken shortly after dosing the cells with the biologically active agent.

The parameter may be an indicator of biological activity or a biologically significant parameter, as previously described. The parameter may be an indicator of metabolic activity of the cells.

The measurement may be taken with the optical sensor. Thus, in some embodiments, the method may comprise positioning the microchannel cell culture device on a platform configured to support the bottom surface of the microchannel cell culture device. The method may comprise coupling the light source to a surface of the platform opposite the microchannel cell culture device. The light source may be configured to direct light towards the plurality of optical sensors. The method may comprise taking the measurement with the optical sensor and transmitting the data to a meter and/or controller or computing device.

The method may comprise after a pre-determined amount of time, taking a second measurement of the parameter within each tissue modeling environment. The method may comprise calculating a rate of change of the parameter to determine the cell biological activity of each cell type or cells within each tissue modeling environment responsive to dosing with the biologically active agent.

The method may comprise imaging the dosed cells. For example, the method may comprise imaging the dosed cells to confirm viability of the cells. The dosed cells may be imaged shortly after dosing. The dosed cells may be imaged after the pre-determined amount of time.

In accordance with one exemplary embodiment, there is provided a method of measuring oxygen consumption of cells. The method may comprise seeding the cells onto at least one tissue modeling environment of a microchannel cell culture device, as disclosed herein. The method may comprise perfusing the cells with cell culture media. The method may comprise incubating the cells for a predetermined period of time. The method may comprise imaging the seeded cells. For example, the method may comprise imaging the seeded cells to confirm viability of the cells.

The method may comprise introducing an oxygen rich fluid into the at least one seeded tissue modeling environment. The oxygen rich fluid may be an air saturated fluid. For example, the oxygen rich fluid may be 21% air saturated fluid. The oxygen rich fluid may be air saturated cell media. The oxygen rich fluid may be air saturated phosphate buffered saline (PBS).

The method may comprise measuring a first oxygen concentration within the at least one seeded tissue modeling environment. The measurement may be taken with the optical sensor, as previously described.

The method may comprise reducing flow rate of the oxygen rich fluid to induce a static environment within the at least one seeded tissue modeling environment. The method may comprise, after a pre-determined amount of time, measuring a second oxygen concentration within the at least one seeded tissue modeling environment. The method may comprise determining the oxygen consumption rate of the cells. For instance, the oxygen consumption rate of the cells may be calculated by the change in oxygen concentration between the first and second measurement.

The method may comprise imaging the cells. For example, the method may comprise imaging the cells to confirm viability of the cells. The cells may be imaged before or after the pre-determined amount of time has elapsed.

According to one aspect, the disclosure relates to an apparatus that includes a well plate, which includes a plurality of structural layers and a membrane. The membrane separates two structural layers and the well plate defines an array of tissue modeling environments. Each tissue modeling environment includes a first fluid reservoir, a second fluid reservoir, a third fluid reservoir, and a fourth fluid reservoir, with each fluid reservoir configured to hold a column of fluid. Each tissue modeling environment also includes a first microchannel fluidically coupling the first fluid reservoir to the second fluid reservoir, and a second microchannel fluidically coupling the third fluid reservoir to the fourth fluid reservoir, wherein a portion of the first microchannel overlaps at least a portion of the second microchannel across the membrane. In some implementations, the fluid reservoirs of the array or tissue modeling environments are arranged to correspond to the arrangement of wells of a standard 96 well or 384 well plate.

In some implementations, the apparatus further includes a pump assembly. For each tissue modeling environment the pump assembly includes a first output for pumping a first fluid into the first fluid reservoir, a first intake for pumping the first fluid out of the second fluid reservoir, a second output for pumping a second fluid into the third fluid reservoir, and a second intake for pumping the second fluid out of the fourth fluid reservoir. In some implementations, the first intake is coupled to the first output and the second intake is coupled to the second output for each tissue modeling environment. The first or second intake of the pump assembly for at least one tissue modeling environment may be coupled to the first or second output of the pump assembly for a different tissue modeling environment. The pump assembly is configured such that the first fluid flows through the first microchannel with a first flow rate and the second fluid flows through the second microchannel with a second flow rate, different from the first flow rate. In some implementations, the pump assembly is configured to control the first flow rate and the second flow rate for each tissue modeling environment. The pump assembly may also include an actuator. The actuator is configured to induce fluid flow through the pump assembly for a plurality of the tissue modeling environments. The pump assembly may also include at least one separate actuator for independently inducing fluid flow through each respective tissue modeling environment. In some implementations, the actuator is an electromagnetic actuator or a hydraulic actuator.

In some implementations, the plurality of structural layers include a first structural layer, a second structural layer, and a third structural layer, wherein the membrane separates the second structural layer and the third structural layer. The first structural layer includes the fluid reservoirs of the tissue modeling environments. The second structural layer defines the first microchannels of the tissue modeling environments, and the third structural layer defines the second microchannels of the tissue modeling environments in the array of tissue modeling environments.

In some implementations, the plurality of structural layers includes a first structural layer and a second structural layer, wherein the membrane separates the first structural layer and the second structural layer, and the first structural layer defines the fluid reservoirs and the first microchannels of the tissue modeling environments in the array of tissue modeling environments.

In some implementations, the first microchannels or the second microchannels of the tissue modeling environment in the array of tissue modeling environments includes a hydraulic resistor including one or more microchannel restrictors.

In some implementations, one or more cells are attached to a first side or a second side of the membrane in each tissue modeling environment. For example, one or more cells attached to the first side of the membrane may be renal proximal epithelial cells and one or more cells attached to the second side of the membrane may be endothelial cells. In some implementations, the portion of the first microchannel overlapping the second microchannel across the membrane is about 1.0 mm to 30 mm in length, 100 μm to 10 mm in width, and 0.05 mm to 1 mm in depth. In some implementations, the membrane is a track-etched polycarbonate or polyester membrane.

In some implementations, the first microchannel and the second microchannel are defined in an embossed hard plastic. The embossed hard plastic may be a cyclic olefin copolymer (COC), fluorinated ethylene propylene (FEP), polymethylpentene (PMP), polyurethane, polystyrene or polysulfone. In some implementations, the first microchannel and the second microchannel are formed from a stack of through-cut layers.

In some implementations, the apparatus further comprises one or more sensor components in each tissue modeling environment. The sensor components may be optical sensors or electrodes.

According to one aspect, the disclosure relates to a method for modeling tissue. The method includes providing a well plate including a plurality of structural layers and a membrane. The membrane separates the two structural layers. The well plate also defines an array of tissue modeling environments. Each tissue modeling environment includes a first fluid reservoir, a second fluid reservoir, a third fluid reservoir and a fourth fluid reservoir, with each fluid reservoir configured to hold a column of fluid. Each tissue modeling environment also includes a first microchannel fluidically coupling the first fluid reservoir to the second fluid reservoir, and a second microchannel fluidically coupling the third fluid reservoir to the fourth fluid reservoir. At least a portion of the first microchannel overlaps at least a portion of the second microchannel across the membrane. The method for modeling tissue modeling environments also includes seeding a first cell type into the first microchannel of each tissue modeling environment, and seeding a second cell type into the second microchannel of each tissue modeling environment. In some implementations, the first cell type may include epithelial cells and the second cell type comprises microvascular cells. The method for modeling tissue modeling environments also includes applying a first feeder flow to the first cell type in the first microchannel of each tissue modeling environment, and applying a second feeder flow to the second cell type in the second microchannel of each tissue modeling environment. In some implementations, the first feeder flow has a first fluid flow rate and the second feeder flow has a second fluid flow rate, different than the first fluid flow rate.

In some implementations, the method for modeling tissue modeling environments also includes introducing a biologically active agent to the array of tissue modeling environments, and measuring the effect of the biologically active agent on the first type of cells or the second type of cells. In some implementations, the biologically active agent also includes introducing different amounts of the biologically active agent into at least two of the tissue modeling environments.

In some implementations, measuring the effect of the biologically active agent on the first or second type of cells includes measuring the effects of the introduction of biologically active agent in different tissue modeling environments having different fluid flow rates.

In some implementations, the method for modeling tissue modeling environments also includes changing a fluid flow rate through the first or the second microchannels of at least one the tissue modeling environment to replicate a hypoxic condition and then measuring the impact of the replicated hypoxic condition on the first or second cell types in at least one tissue modeling environment.

Systems and methods for providing an array of tissue modeling environments with dynamic control of fluid flow are disclosed herein. The disclosure describes a cell culture platform with arrays of wells that are fluidically coupled by microchannel structures. A dynamically controlled flow of fluid, when administered through the wells and microchannels, interacts with cells grown within the microchannels. The fluid flow can be used to condition the cells, maintain their growth, perfuse the tissue, supply media/fluids, seed cells, administer mechanical forces/stresses, introduce therapeutic molecules and/or collect samples. The present disclosure also provides systems and methods for providing an array of integrated real time sensors to enable the characterization of tissue conditions and tissue response to exposure to such fluid flows.

In some implementations, systems and methods according to the present disclosure may provide a tissue culture optimization tool in which each tissue modeling environment may be subjected to unique conditions to optimize cell cultures within the tissue modeling environment. In some implementations, systems and methods according to the disclosure may provide a drug screening array in which the tissue in each tissue modeling environment can be screened against a different drug and/or a different dose of the same drug. In some implementations, the systems and methods according to the disclosure can provide drug delivery analysis in which the fluid flow of each tissue unit can be configured to simulate distribution and delivery of a drug in the bloodstream to a tissue. In some implementations, the systems and methods according to the disclosure can provide disease modeling in which each tissue modeling environment or groups of tissue modeling environments can be subjected to unique conditions to model varying disease states.

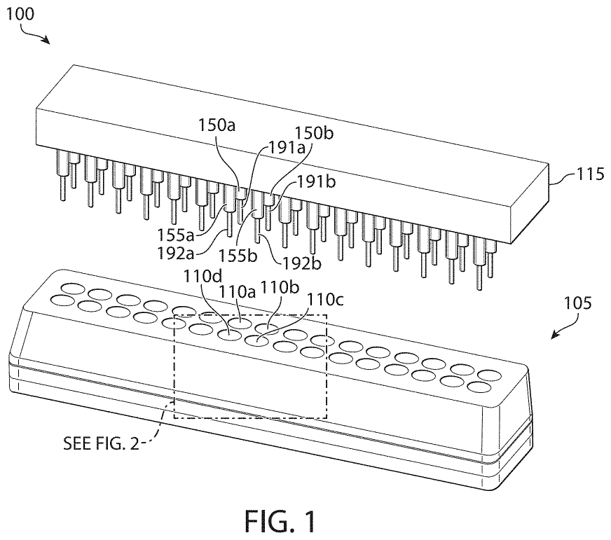

FIG. 1 illustrates an apparatus 100. The apparatus 100 includes a cell culture platform 105 and a pump assembly 115 for providing an array of tissue modeling environments with controlled fluid flow. The cell culture platform 105 includes an array of tissue modeling environments. Each tissue modeling environment includes a group of fluid reservoirs fluidically coupled by a pair of microchannel structures that are separated by a membrane. Each tissue modeling environment includes a group of fluid reservoirs such as a first fluid reservoir 110a, a second fluid reservoir 110b, a third fluid reservoir 110c and a fourth fluid reservoir 110d (generally referred to as fluid reservoirs 110). The fluid reservoirs 110 are each configured to hold a vertical column of fluid. In some implementations, the placement and spacing of the fluid reservoirs 110 of the cell culture platform 105 closely match the placement and spacing of the wells in a standard well plate such as a 96 well or a 384 well plate. This configuration of fluid reservoirs enables the cell culture platform 105 to be compatible with standard industry equipment, such as micropipettes and imaging systems, which are designed for the well configurations of standard well plates.

The pump assembly 115 also includes a plurality of fluid input valves such as a first input valve 150a and a second input valve 150b, as well as a plurality of fluid output valves such as a first fluid output valve 155a and a second fluid output valve 155b. The pump assembly also includes a plurality of input valve sippers such as a first input valve sipper 191a and a second input valve sipper 191b and a plurality of output valve sippers such as a first output valve sipper 192a and a second output valve sipper 192b. When the pump assembly 115 is positioned above the cell culture platform 105, the first and second input valve sippers 191a and 191b and the first and second output valve sippers 192a and 192b are inserted into the columns of fluid in the fluid reservoirs 110. The flow rate through a microchannel fluidically coupling a pair of fluid reservoirs depends partially on the relative fluid heights in the respective fluid reservoirs 110 as maintained by the pumping of fluid from one fluid reservoir to a different fluid reservoir.

The volume of a fluid reservoir may be about 60 μL but may be between about 50 to 115 μL in some implementations. The height of a fluid reservoir may be 11.38 mm but can between about 9 mm to 12 mm to in some implementations. The diameter of the top of a fluid reservoir may be about 3.7 mm but can between about 2.7 mm to 4.7 mm to in some implementations. In some implementations, the fluid reservoir pitch can be about 4.5 mm by 4.5 mm and can have a tolerance of 0.05 in diameter. In some implementations, the fluid reservoirs are tapered towards the bottom to ensure that cells introduced into the fluid reservoirs move through the fluid reservoirs to the membrane 140.

The dimensions of the fluid reservoirs may be altered to achieve a fluid volume to cell count ratio similar to a physiological system to limit any dilution effect. However, in vitro systems can be limited by both the oxygen carrying capacity of the media as well as missing nutrients, hormones, and other secreted factors present in physiological systems. The typical per cell fluid volumes for human physiology may be about 15 pL per cell. A typical 96 well plate may hold about 2000 pL per cell, a typical 24 well plate may hold about 900 pL per cell, and a typical 12 well plate may hold about 6000 pL per cell.

In some implementations, system and methods according to the present disclosure may provide a desirable fluid volume to cell count range of about 100 to 1200 pL per cell, assuming 2,700 cells per mm².

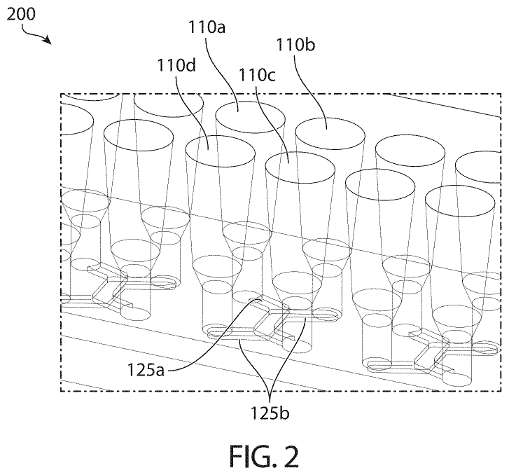

As indicated above, each tissue modeling environment of the cell culture platform 105 also includes a pair of microchannel structures. Each microchannel structure is generally configured to fluidically couple a pair of fluid reservoirs in a tissue modeling environment. FIG. 2 illustrates a magnified view of a tissue modeling environment provided by the example apparatus 100 illustrated in FIG. 1. Each tissue modeling environment of cell culture platform 105 includes a first microchannel 125 a and a second microchannel 125 b (generally referred to as microchannels 125). The first microchannel 125 a fluidically couples the first fluid reservoir 110 a to the third fluid reservoir 110 c. The second microchannel 125 b fluidically couples the second fluid reservoir 110 b to the fourth fluid reservoir 110 d. A portion of the first microchannel 125 a overlaps and runs parallel to at least a portion of the second microchannel 125 b.

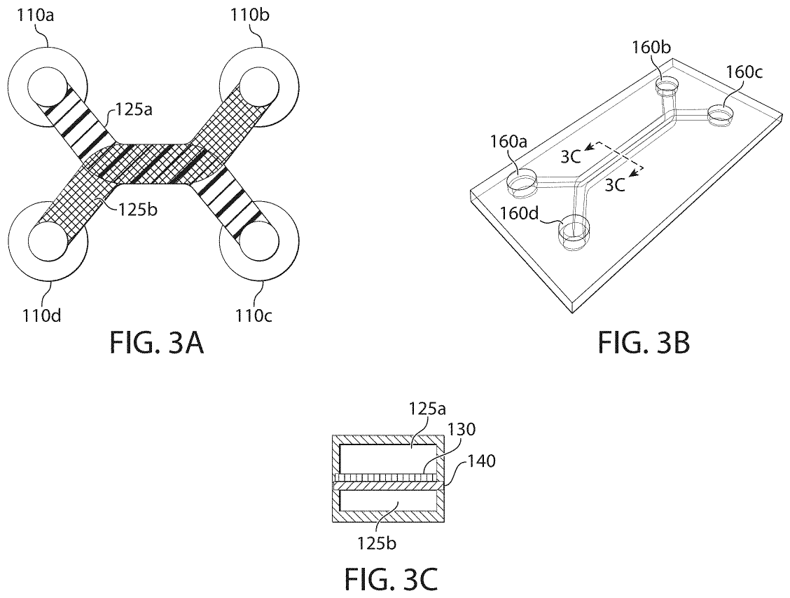

FIG. 3A illustrates a top down view of the tissue modeling environment illustrated in FIG. 2. The first microchannel 125 a fluidically couples the first fluid reservoir 110 a to the third fluid reservoir 110 c. The second microchannel 125 b fluidically couples the second fluid reservoir 110 b to the fourth fluid reservoir 110 d. A portion of the first microchannel 125 a overlaps and runs parallel to at least a portion of the second microchannel 125 b. In some implementations, the microchannels 125 may be fabricated using an embossed hard plastic, thus eliminating disadvantages of microfluidic devices fabricated using soft polymer materials such as PDMS.

FIG. 3B and FIG. 3C illustrates perspective views and cross sectional views, respectively, of the tissue modeling environment illustrated in FIG. 2. In FIG. 3B, the first microchannel 125 a is coupled with a first port 160 a and a third port 160 c, and the second microchannel 125 b is coupled with a second port 160 b and a forth port 160 d (generally referred to as the ports 160). The ports 160 couple the microchannels 125 to the fluid reservoirs 110. As previously indicated, each tissue modeling environment of the cell culture platform 105 also includes a membrane 140. A portion of the first microchannel 125 a overlaps and runs parallel to a portion of the second microchannel 125 b across the membrane 140. In some implementations, the membrane 140 may have cells 130 attached to it forming living tissue. In some implementations, the membrane 140 may be a semi-permeable membrane with a porosity between about 5 to 90 percent. In some implementations, the membrane 140 may be a semi-permeable track etched membrane with a thickness between about 10 nm to 10 microns. In some implementations the membrane 140 may be a non-permeable membrane. In some implementations, the membrane 140 may be a tensioned membrane. In some implementations, the membrane 140 may be a non-tensioned membrane that includes fluorinated ethylene propylene (FEP). In some implementations, the membrane 140 may include a scaffolding of polycarbonate, polyethylene terephthalate (PET) or polyamide. In some implementations, the membrane 140 may include a hydrogel, gel or cross linked elastomer.

In some implementations, cells of the same cell type or cells of different cell types may be attached to each side of the membrane 140 or the walls of the microchannels to create a co-culture. In some implementations, renal proximal epithelial tissue may be seeded on the apical or top surface of the membrane 140 while endothelial cells may be seeded on the bottom surface of the membrane 140 to approximate the in vivo structure of the renal tubule. In some implementations, intestinal epithelial cells may be seeded on the top surface of the membrane 140 and endothelial cells may be seeded on the bottom surface of the membrane 140 to approximate the in vivo structure of gastrointestinal tissue. In some implementations, airway epithelial cells may be seeded on the top surface of the membrane 140 and endothelial cells may be seeded on the bottom surface of the membrane 140 to approximate the in vivo structure of airway tissue, lung tissue, or tracheobronchial tissue. In some implementations, tumor cells may be seeded on the top surface of the membrane 140 and endothelia cells may be seeded on the bottom surface of the membrane 140 to approximate the in vivo structure of a tumor environment. In some implementations, hepatocyte cells may be seeded on the top surface of the membrane 140 and endothelial cells may be seeded on the bottom surface of the membrane 140 to approximate the in vivo structure of a liver sinusoid. In some implementations, hepatocyte cells may be seeded on the top surface of the membrane 140 and stellate cells and Kupffer cells may be seeded on the bottom surface of the membrane 140 to approximate the in vivo structure of liver tissue. In some implementations, pericytes or smooth muscle cells may be seeded on the top surface of membrane 140 and endothelial cells may be seeded on the bottom surface of the membrane 140 to approximate the in vivo structure of vascular tissue. In some implementations, oral keratinocytes or fibroblasts may be seeded on the top surface of the membrane 140 and endothelial cells may be seeded on the bottom surface of the membrane 140 to approximate the in vivo structure of oral tissue, for example, gum tissue. In some implementations, epidermal keratinocytes or fibroblasts may be seeded on the top surface of the membrane 140 and endothelial cells may be seeded on the bottom surface of the membrane 140 to approximate the in vivo structure of skin tissue. In some implementations, central nervous system cells may be seeded on the top surface of the membrane 140 and endothelial cells may be seeded on the bottom surface of the membrane 140 to approximate the in vivo structure of a blood brain barrier tissue. In some implementations, syncytiotrophoblasts may be seeded on the top surface of membrane 140 and endothelial cells may be seeded on the bottom surface of membrane 140 to approximate the in vivo structure of placental barrier tissue. In some implementations, immune cells, such as T cells, may be included in any cell combination to approximate an in vivo tissue response to an immune interaction component.

In some implementations, one or more portions of a tissue modeling environment may include a cell-phobic coating to selectively prevent cells introduced into the tissue modeling environment from adhering to the coated areas. In some implementations, portions of a tissue modeling environment may include a cell-binding coating to selectively bind cells introduced into the tissue modeling environment to the coated portions. In some implementations, the cell-binding coating may be used in place of or in conjunction with the cell-phobic coating. In some implementations, the cell-phobic coating and the cell-binding coating may include patterns, cell adhesion molecules (CAMs) or nanotopographic patterns. In some implementations, at least one surface of the membrane 140 includes a topographical pattern. In some implementations, the topographical pattern may be a nanotopographical pattern. In some implementations, the topographical pattern on at least a portion of at least one surface of the membrane 140 is selected to promote increased adhesion of cells to at least one surface of the membrane 140, as described in U.S. application Ser. No. 13/525,085, the entirety of which is incorporated herein by reference. For example, in some implementations, the design of the topographic surface allows close control of cells grown atop the substrate. In some implementations, the topographic surface, along with additional flow channel parameters such as channel height, channel cross-sectional area, and flow rate, can be used to create highly controlled in vitro conditions that closely mimic the in vivo environment of specific cells types. For example, in some implementations, a pattern of grooves and ridges, like an extracellular matrix, may causes kidney cells to lengthen and align themselves parallel to the ridges, encourage cell-to-cell junctions, and promote the adhesion of the cells to the surface. In some implementations, the membrane surface can have grooves and ridges that are narrower than the cells. In some implementations, the grooves and ridges are approximately the same width, although they do not have to be.

As shown in FIGS. 2 and 3A-3C a portion of the first microchannel 125 a overlaps and runs in parallel to at least a portion of the second microchannel 125 b. In some implementations, one or more portions of the first microchannel 125 a and the second microchannel 125 b may overlap one or more portions of the second microchannel 125 b without the channels running parallel to one another at the overlap. For example, in some implementations, at least one of the first microchannel 125 a and the second microchannel 125 b may have a serpentine shape and may cross each other at various points.

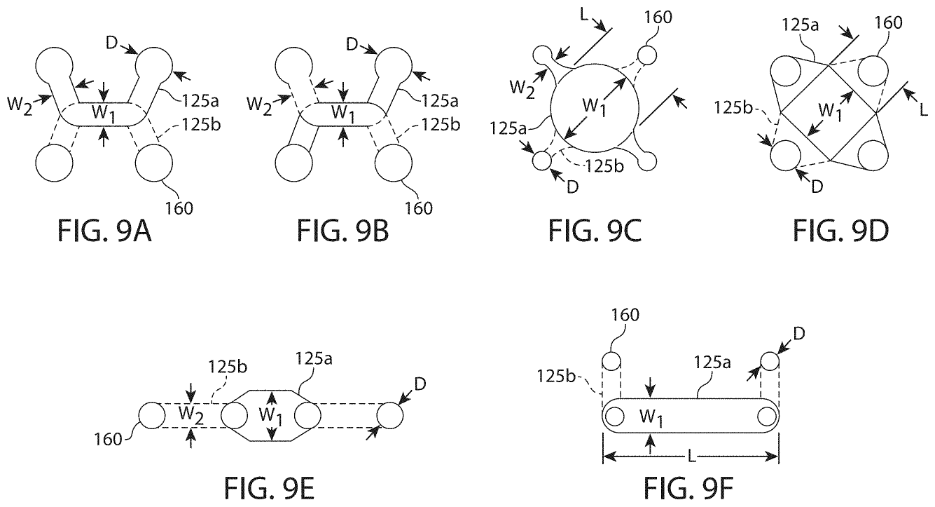

In some implementations, the microchannels 125 may be between about 1 to 30 mm in length. In some implementations, the microchannels 125 may be between about 100 μm to 10 mm in width. In some implementations, the microchannels 125 may be between about 0.05 mm to 1 mm in depth. FIGS. 9A-9F below further illustrate example implementations of the microchannels 125.

FIGS. 9A-9F show example implementations of the microchannels 125. FIGS. 9A-9F represent a variety of channel configurations that can be built into the platform 105. Each structure accomplishes one or more design goals that enhance the functionality of the well plate platform for a variety of corresponding use cases depending on the geometrical and biophysical requirements of particular tissue models being cultured. The solid lines in the Figures represent the first microchannel 125 a and dashed lines represent second microchannel 125 b. As previously mentioned, the first microchannel 125 a fluidically couples the first reservoir 110 a to the third reservoir 110 c. The second microchannel 125 b fluidically couples the second reservoir 110 b to the fourth reservoir 110 d. Each of each microchannel has a port 160 connecting the microchannel 125 to a corresponding fluid reservoir 110.

FIG. 9A illustrates a top down view of an example implementation of microchannels 125 in a cis orientation. In a cis orientation, the ports 160 for each microchannel are on the same side of the channel. In FIG. 9A, the width W₁ of the overlapping portions of the microchannels may be about 1.0 to 1.5 mm and the width W₂ of the non-overlapping portions of the microchannels may be about 0.75 to 1.0 mm. In some implementations, the depth (not shown) of the microchannels may be about 100 to 200 μm. In some implementations, the depth (not shown) of the microchannels may be about 100 to 300 μm. In some implementations, the diameter D of the ports 160 may be about 100 to 300 μm. In some implementations, the diameter D of the ports 160 may be about 500 to 1500 μm. The length of the overlapping portions of the microchannels channels may be between 6 to 8 mm, for example 7 mm.

FIG. 9B illustrates a top down view of an example implementation of microchannels 125 in a trans orientation. In a trans orientation, the ports 160 for each microchannel are on opposite sides of the channel. In FIG. 9B, the width W₁ of the overlapping portions of the microchannels may be about 1.0 to 1.5 mm and the width W₂ of the non-overlapping portions of the microchannels may be about 0.75 to 1.0 mm. In some implementations, the depth (not shown) of the microchannels may be about 100 to 200 μm. In some implementations, the depth (not shown) of the microchannels may be about 100 to 300 μm. In some implementations, the diameter D of the ports 160 may be about 100 to 300 μm. In some implementations, the diameter D of the ports 160 may be about 500 to 1500 μm. The length of the overlapping portions of the microchannels channels may be between 6-8 mm, for example 7 mm.

FIG. 9C illustrates a top down view of an example implementation of microchannels 125. The implementation of FIG. 9C maximizes the overlapping area of the microchannels 125, where the overlap area may be between 80% to 85% of total microchannel area, for example 82%. The width W₁ of the overlapping portions of the microchannels may be about 3 to 4.25 mm. The width W₂ of the non-overlapping portions of the microchannels may about 0.5 to 1 mm. The length of the microchannel L may be about 3.0 to 4.25 mm. The depth (not shown) may be about 100 to 300 μm. The diameter D of the ports 160 may be about 0.5 to 1.5 mm.

FIG. 9D illustrates a top down view of an example implementation of microchannels 125. FIG. 9D maximizes cell culture area of the microchannels 125, where the cell culture area may be between 20 mm² and 30 mm², for example 25.4 mm². The width W₁ of the overlapping portions may be about 3.0 to 4.25 mm. The length L may be about 3 to 4.25 mm and the depth (not shown) may be about 100 to 300 μm. The diameter D of the ports 160 may be about 0.75 to 1.5 mm.

FIG. 9E illustrates a top down view of an example implementation of microchannels 125. The implementation of FIG. 9E creates a uniform flow field in both upper and lower microchannels 125. The width W₁ of the overlapping portions of the microchannels may be about 1.0 to 3.5 mm. The width W₂ of the non-overlapping portions of the microchannels may be 0.5 to 1.5 mm. In some implementations, the depth (not shown) of the microchannels may be about 100 to 250 μm. In some implementations, the depth (not shown) of the microchannels may be about 100 to 300 μm. The diameter D of the ports 160 may be about 1.0 to 1.5 mm. FIG. 9E may be orientated in a linear orientation formed from four ports 160 arranged in a single row or column of a well plate instead of a 2×2 grouping of wells.

FIG. 9F illustrates a top down view of an example implementation of microchannels 125. FIG. 9F creates a high overlap area of about 70% to 80%. In some implementations, the overlap area may be about 76%. The upper channel has a uniform flow field because the ports are linearly connected to the microchannel 125 a, compared to microchannel 125 b where the ports are located at an angle to microchannel 125 b. The width W₁ of the microchannel overlap may be about 3.0 to 4.25 mm. The length L may be about 6.0 to 7.5 mm. The depth (not shown) may be about 100 to 300 μm. The port diameter D may be about 0.5 to 1.5 mm.

In some implementations, the fluid flow through the microchannels 125 may be used to condition the cells attached to the membrane 140, maintain their growth, perfuse the tissue, supply media/fluids, seed cells, introduce therapeutic molecules and collect samples. In some implementations, fluid pumped through the microchannels 125 may include suspended cells, for example blood cells. In some implementations, the flow rates and media composition through a tissue modeling environment may be different in each of the microchannels 125 while interacting through the tissue attached to the membrane 140. In some implementations, the flow rate through the first microchannel 125 a or the second microchannel 125 b or both microchannels 125 a and 125 b may be zero.

The difference in fluid column height between a pair of fluid reservoirs causes a gravity fed fluid flow through the first and second microchannels 125 a and 125 b. A fluid flow through both the first microchannel 125 a and the second microchannel 125 b maintains a pressure gradient across the membrane 140 within the microchannels 125. In some implementations, the fluid flows through the microchannels 125 may be used to administer mechanical forces and stresses across the membrane 140. The shear rate applied to the membrane 140 within the microchannels 125 is determined by the flow rate through the microchannels 125 and their dimensions as well as the geometry of the overlapping portion of the first microchannels 125 a and the second microchannel 125 b. In some implementations, a hydraulic resistance in the microchannels 125 may be fixed or varied through the use of channel restrictors that are actuated. The advantage of having variable hydraulic resistance in the microchannels 125 is that the shear stress applied to the membrane 140 within the microchannels 125 may be varied in a customized, “plug and play” manner in contrast to a cell culture platform with fixed microchannel dimensions. In some implementations, additional time-varying controls through the actuation of pumps and valves or by manipulation of distensible walls of the microchannels 125 can be used to introduce pulsatile or time-varying shear rates upon cultured cell populations within the microchannels 125. In some implementations, the cell culture platform 105 and/or pump assembly 115 may include hydraulic capacitive or compliant elements.

When a pair of fluid reservoirs are fluidically coupled by a microchannel, a difference in height between a column of fluid in each fluid reservoir causes a gravity fed fluid flow through the microchannel. Controlling the difference in fluid column height between a pair of fluid reservoirs controls the rate of the fluid flow through a microchannel. A difference in height between the columns of fluid in a pair of fluid reservoirs is achieved by introducing fluid into one fluid reservoir and/or removing fluid from another fluid reservoir through a pathway other than the microchannel. A desired fluid flow rate through a microchannel is produced by maintaining an approximately constant difference in fluid column height between the pair of fluid reservoirs by introducing and removing fluid from their fluid columns at a rate that is equal to the desired fluid flow rate. A controlled fluid flow, when administered through the fluid reservoirs and the microchannel structures of a tissue modeling environment, interacts with cells attached to the membrane within the microchannel structures.

Flow rates can be determined from a number of different biological requirements including transport, reaction kinetics, and mechanical effects (e.g. shear). Transport can be calculated from the convective diffusion equation, known to persons of ordinary skill in the art. In the case where transport is an important biological design principle, e.g. hepatocytes, matching the in vitro model transport regime to the physiological transport regime can be accomplished using the Peclet number, a dimensionless parameter that indicates the ratio of convective to diffusive transport, where

$$P e = \frac{U L}{D}$$

wherein L is the length, U is the velocity magnitude, and D is a characteristic diffusion coefficient. The channel geometry will set the length, L, the diffusing species and media will determine the diffusion coefficient, D, and the flow rate can then be determined from the channel geometry and the mean velocity, U. For transport without reaction, the Peclet number is an appropriate scaling parameter. For example, in vivo estimates of the Peclet number in the blood compartment of the liver sinusoid are between 2 and 10.

Fluid flow within microchannels 125 creates shear stress on the membrane 140. Shear stress can be calculated by solving the Naiver Stokes equation of fluid flow, but has been reduced to analytical solutions known to persons of ordinary skill in the art for simplified geometries. For the cases where flow pulsatility is an important parameter, estimates can be made using the system capacitance and resistance. For a pressure driven flow from an open reservoir of constant cross-section, the capacitance and resistance are given respectively by:

$$C = \frac{Well Surface Area}{ρ g}$$

wherein C is the fluidic capacitance, ρ is the fluidic density, and g is the gravity constant;

$$9.81 \frac{m}{{s}^{2}} and R = \frac{Δ P}{Q}$$

where R is the system fluidic resistance, ΔP is the pressure difference and Q is the flow rate. The fluidic time constant is given by the product of the fluidic capacitance, C, and the fluidic resistance, R. To minimize flow pulsatility, the system may be designed such that the pump cycle is significantly less than the fluidic time constant. The change in flow rate between cycles may be estimated by:

Q=Q₀e^{-t/RC}

where t is time and Q₀ is the flow rate at t=0.

These equations together with the system constraints, including biological constraints, fabrication, materials, etc., can be combined to create a solution envelope of possible geometrics satisfying all constraints.

As previously indicated, the example apparatus 100 in FIG. 1 also includes a pump assembly 115. The pump assembly 115 is generally configured to provide controlled fluid flow to each tissue modeling environment of the cell culture platform 105 by pumping fluid into and out of the tissue modeling environment through a plurality of fluid input valves and fluid output valves. The pump assembly 115 includes a plurality of fluid input valves such as a first fluid input valve 150 a and a second fluid input valve 150 b (generally referred to as the fluid input valves 150). The pump assembly 115 also includes a plurality of fluid output valves such as a first fluid output valve 155 a and a second fluid output valve 155 b (generally referred to as the fluid output valves 155). The fluid input valves and the fluid output valves are check valves. The fluid input valves are generally configured to intake fluid flow into the pump assembly 115 while the fluid output valves are generally configured to output fluid flow out of the pump assembly 115.

The pump assembly 115 includes a plurality of input valve sippers and a plurality of output valve sippers. The input valve sippers and the output valve sippers are generally configured to enable the fluid input valves 150 and the fluid output valves 155 to be in fluid communication with the fluid reservoirs 110. A first fluid input valve 150 a is coupled to a first input valve sipper 191 a, a second fluid input valve 150 b is coupled to a second input valve sipper 191 b, a first fluid output valve 155 a is coupled to a first output valve sipper 192 a and a second fluid input valve 155 b is coupled to a second output valve sipper 192 b. When the pump assembly 115 is positioned above the cell culture platform 105, the first and second input valve sippers 191 a and 191 b and the first and second output valve sippers 192 a and 192 b are inserted into the columns of fluid in the fluid reservoirs 110 at different depths. The flow rate through a microchannel fluidically coupling a pair of fluid reservoirs depends partially on the depth of the input valve sippers and the output valve sippers in the fluid reservoirs 110.

When the pump assembly 115 is positioned above the cell culture platform 105, the fluid input valves 150 and the fluid output valves 155 of the pump assembly 115 are in fluid communication with the fluid reservoirs 110. The first fluid input valve 150 a and the first input valve sipper 191 a and the second fluid input valve 150 b and the second input value sipper 191 b are each in fluid communication with a column of fluid in the first fluid reservoir 110 a and the second fluid reservoir 110 b, respectively. The first fluid output valve 155 a and the first output value sipper 192 a and the second fluid output valve 155 b and the second output value sipper 192 b are each in fluid communication with a column of fluid in the fourth fluid reservoir 110 d and the third fluid reservoir 110 c, respectively. The pump assembly 115 pumps a fluid out of the first fluid reservoir 110 a through the first fluid input valve 150 a and the first input valve sipper 191 a. The pump assembly 115 pumps a fluid out of the second fluid reservoir 110 b through the second fluid input valve 150 b and the second input valve sipper 192 b. The pump assembly 115 pumps fluid into the forth fluid reservoir 110 d through the first output value 155 b and the first output value sipper 192 a. The pump assembly pumps fluid into the third fluid reservoir 110 c through the second fluid output valve 155 b and the second output valve sipper 192 b.

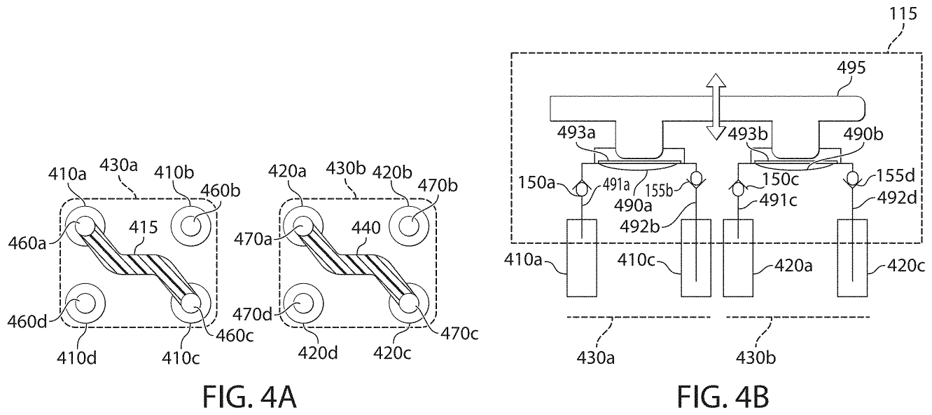

FIG. 4A illustrates a top down view of two adjacent tissue modeling environments 430 a and 430 b (each generally referred to as a tissue modeling environment 430) of the cell culture platform 105 in FIG. 1. Each of the tissue modeling environments 430 includes a plurality of fluid reservoirs. The first tissue modeling environment 430 a includes a first fluid reservoir 410 a, a second fluid reservoir 410 b, a third fluid reservoir 410 c and a fourth fluid reservoir 410 d. The first fluid reservoir 410 a and the third fluid reservoir 410 c are fluidically coupled by a first microchannel 415. While not shown in FIG. 4A, the second fluid reservoir 410 b and the fourth fluid reservoir 410 d are also fluidically coupled by a microchannel. The first tissue modeling environment 430 a also includes a plurality of ports. The first tissue modeling environment includes a first port 460a, a second port 460b, a third port 460c, and a forth port 460d. The first port 460a couples the first microchannel 415 to the first fluid reservoir 410a. The second port 460b couples the second microchannel to the second fluid reservoir 410b. The third port 460c couples the first microchannel 415 to the third fluid reservoir 410c, and the forth port 460d couples the second microchannel to the forth fluid reservoir 410d. The second tissue modeling environment 430b includes a first fluid reservoir 420a, a second fluid reservoir 420b, a third fluid reservoir 420c and a fourth fluid reservoir 420d. The first fluid reservoir 420a and the third fluid reservoir 420c are fluidically coupled by a first microchannel 440. While not shown in FIG. 4A, the second fluid reservoir 420b and the fourth fluid reservoir 420d are also fluidically coupled by a microchannel. The second tissue modeling environment 430b also includes a plurality of ports. The second tissue modeling environment 430b includes a first port 470a, a second port 470b, a third port 470c, and a forth port 470d. The first port 470a couples the first microchannel 440 to the first fluid reservoir 420a. The second port 470b couples the second microchannel to the second fluid reservoir 420b. The third port 470c couples the first microchannel 440 to the third fluid reservoir 420c, and the forth port 470d couples the second microchannel to the forth fluid reservoir 420d.

FIG. 4B illustrates the pump assembly 115 interacting with the first and second tissue modeling environments 430a and 430b shown in FIG. 4A. The first fluid input valve 150a is coupled to a first input sipper 491a and is in fluid communication with a column of fluid in the first fluid reservoir 410a of the first tissue modeling environment 430a. The second fluid input valve 150b (not shown in FIG. 4A or FIG. 4B) of the pump assembly 115 is adjacent to the first fluid input valve 150a. A third fluid input valve 150c is coupled to a third input sipper 491c and in fluid communication with a column of fluid in the first fluid reservoir 420a in the second tissue modeling environment 430b. A fourth fluid input valve 150d (not shown in FIG. 4A or FIG. 4B) of the pump assembly 115 is adjacent to the third fluid input valve 150c.

The pump assembly 115 includes a first fluid output valve 155a (not shown in FIG. 4A or FIG. 4B). The second fluid output valve 155b is coupled to a second output sipper 492b and is in fluid communication with a column of fluid in the third fluid reservoir 410c of the first tissue modeling environment 430a. A third fluid output valve 155c (not shown in FIG. 4A or FIG. 4B) of the pump assembly 115 is adjacent to the second fluid output valve 155b. A fourth fluid output valve 155d is coupled to a fourth output sipper 492a and is in fluid communication with a column of fluid in the third fluid reservoir 420c in the second tissue modeling environment 430b.

The pump assembly 115 further includes an actuator 495. The actuator 495 is generally configured to control a fluid flow through the first tissue modeling environment 430a and a fluid flow the second tissue modeling environment 430b. The actuator 495 pumps fluid flow through the first input valve 150a and the second output valve 155b in the first tissue modeling environment 430a and the third input valve 150c and the fourth output valve 155d in the second tissue modeling environment 430b. The pump assembly further includes a first pump chamber 490a, a second pump chamber 490b (generally referred to as the pump chambers 490), a first pump chamber diaphragm 493a and a second pump chamber diaphragm 493b (generally referred to as the pump chamber diaphragms 493). The pump chamber diaphragms 493 are tensioned. The first pump chamber 490a and the second pump chamber 490b are each generally configured to hold a fluid.

The pump assembly 115 pumps a fluid into the third fluid reservoir 410c and 420c of the first and second tissue modeling environments 430a and 430b. As previously indicated, the pump chamber diaphragms 493 are tensioned and can be depressed by the actuator 495. When the actuator 495 is lowered, it depresses the pump chamber diaphragms 493 into the pump chambers 490 causing fluid in the pump chambers 490 to flow through the second fluid output valve 155b and the fourth fluid output valve 155d (generally referred to as the fluid output valves 155) and into the third fluid reservoirs 410c and 420c of the first and second tissue modeling environments 430a and 430b. As the fluid flows into the third fluid reservoirs 410c and 420c of the first and second tissue modeling environments 430a and 430b produces a difference in fluid column height between the first fluid reservoirs 410a and 420a and the third fluid reservoirs 410c and 420c causing a gravity fed fluid flow through the microchannels 415 and 440.

The pump assembly 115 pumps a fluid out of the first fluid reservoirs 410a and 420a of the first and second tissue modeling environments 430a and 430b. When the actuator 495 retracts from the pump chamber diaphragms 493, the tensioned pump chamber diaphragms 493 return to their rest position, pulling a vacuum on the pump chambers 490, and causing fluid to move out the first fluid reservoirs 410a and 420a of the first and second tissue modeling environments 430a and 430b through the first fluid input valve 150a and the third fluid input valve 150c (generally referred to as the fluid input valves 150) and into the pump chambers 490. As the fluid flows out of the first fluid reservoirs 410a and 420a of the first and second tissue modeling environments 430a and 430b, gravity fed flow causes the fluid column heights of the first fluid reservoirs 410a and 420a and the third fluid reservoirs 410c and 420c to equalize.

In some implementations, a separate actuator may be provided for each tissue modeling environment and may be independently controlled for each tissue modeling environment. In some implementations, the actuators provided for the tissue modeling environments may be independently controlled with electromagnetic actuators. In other implementations, a single actuator may drive fluid flow for multiple tissue modeling environments within a cell culture platform. In other implementations, a single actuator may drive fluid flow for all of the tissue modeling environments within a cell culture platform. In some implementations, the fluid input and output valves may be duckbill style valves, valves composed of asymmetric diffusers or other directionally-biased flow valves. Other implementations may include actuator and fluid valve configurations as described in U.S. Patent Application Publication No. 2016/0220997 titled “Actuated valve or pump for microfluidic devices” filed Feb. 4, 2016, the entirety of which is incorporated herein by reference for all purposes.

In some implementations, the pump assembly 115 is controlled by a controller. In some implementations, the controller outputs actuation and control signals to the actuator for each tissue modeling environment in the array of tissue modeling environments. In some implementations, the controller may include a user interface, by which the user may enter in the desired flow rates for each tissue modeling environment. In some implementations, the controller is further configured to receive, store, and process, sensor data collected by the sensors discussed further below. The results of the sensor data processing can be outputted via the user interface. In some implementations, the controller includes software executing on a general purpose processor configured to provide the above-referenced user interface and to output the above-mentioned control signals.

In some implementations, the pump chamber diaphragms may be non-tensioned and thus are unable to return to a rest position upon retraction of the reservoir. The pump chamber diaphragms in such implementations must be actively pulled back to their non-depressed position. Such pumps can be driven, in some implementations, by pneumatic fluid flow, where introduction of a fluid distends the diaphragm into the pump chamber cavity, and withdrawal of the pneumatic fluid creates a vacuum which actively retracts the diaphragm.

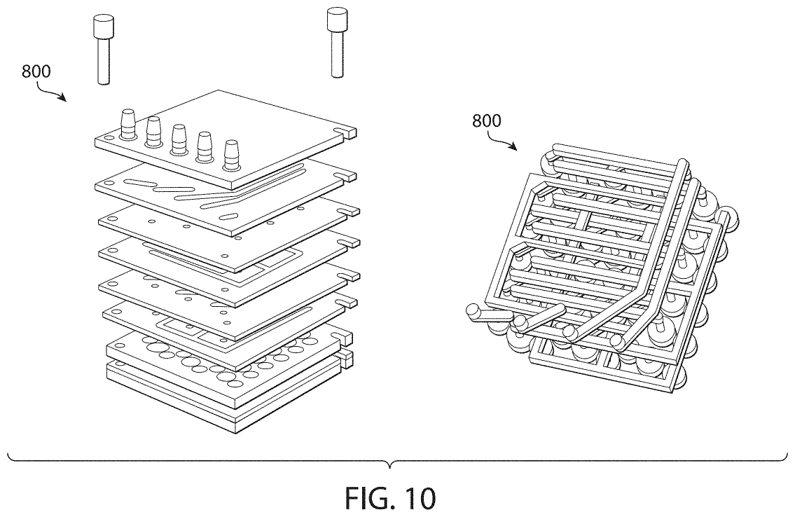

FIG. 10 illustrates two views of an example pneumatic pump assembly 800 suitable for driving pumps with non-tensioned or tensioned membranes. The pneumatic pump assembly 800 includes pneumatic pump lines that provide pneumatic fluid to and from rows of pumps in the pump assembly 800 such that the pumps in those rows act in unison. For example, one row of pumps may control the flow of fluid that passes through the first microchannels 125a of multiple cell culture environments, while another row of pumps may control the flow of fluid that passes through the second microchannels 125b of those cell culture environments. The pump assembly 800 can also include fluid lines for applying bias pressures to passive valves included in the pump assembly 800.

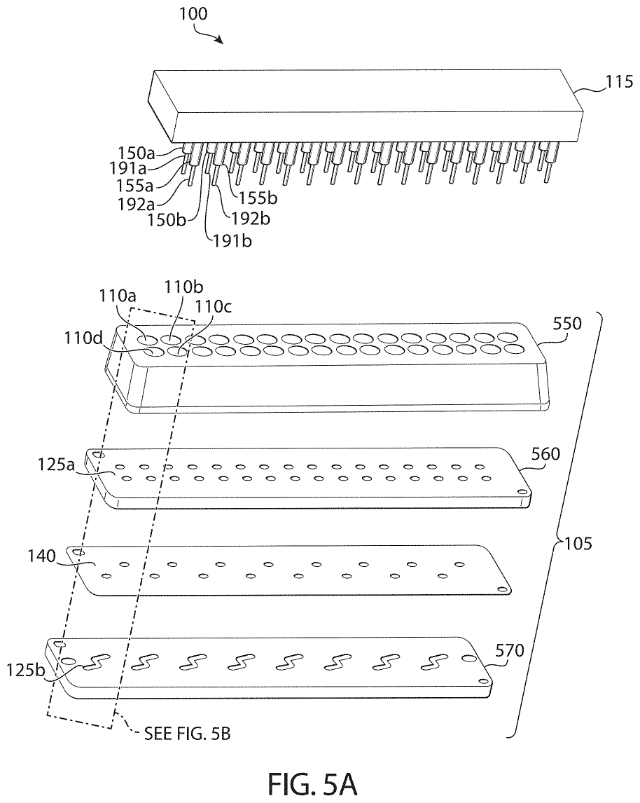

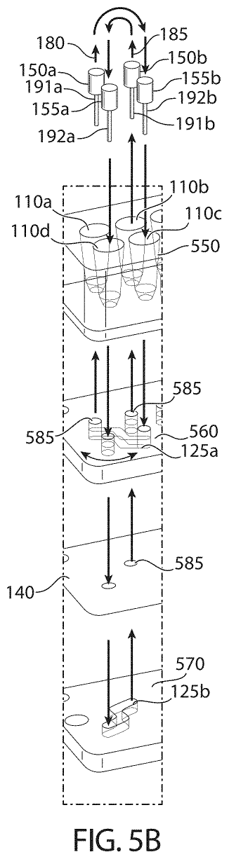

FIG. 5A illustrates an exploded view of the cell culture platform 105 of the example apparatus 100 illustrated in FIG. 1. An array of tissue modeling environments in a cell culture platform is defined by structural layers separated by a membrane. The cell culture platform 105 includes a first structural layer 550 that further includes a plurality of fluid reservoirs 110, a second structural layer 560 and a third structural layer 570 separated by a membrane 140. The fluid reservoirs 110 are each configured to hold a vertical column of fluid. The underside of the second structural layer 560 defines a first set of microchannel structures (shown in FIG. 5B), such as first microchannels 125a. The first microchannels 125a fluidically couple the first fluid reservoirs 110a to the third fluid reservoirs 110c. The third structural layer 570 defines a second set of microchannel structures, such as the second microchannels 125b. The second microchannels 125b fluidically couple the second fluid reservoirs 110b to the fourth fluid reservoirs 110d. A membrane 140 separates the first set of microchannels defined by the second structural layer 560, such as the first microchannel 125a and the second set of microchannels defined by the third structural layer 570 such as the second microchannel 125b. When the first structural layer 550, the second structural layer 560 and the third structural layer 570 are stacked, portions of the first microchannels 125a overlap and run parallel to portions of the second microchannels 125b across the membrane 140. In some implementations, at least one of the first microchannels 125a and the second microchannels 125b may have a serpentine shape and may cross each other at various points. In some implementations, the microchannels 125 may be between about 1 to 30 mm in length. In some implementations, the microchannels 125 may be between about 100 μm to 10 mm in width. In some implementations, the microchannels 125 may be between about 0.05 mm to 1 mm in depth. FIGS. 9A-9F, discussed above, further illustrate example implementations of the microchannels 125.

FIG. 5B illustrates fluid pathways through the structural layers of the example cell culture platform 105 illustrated in FIG. 5A. In FIG. 5B, a gravity fed fluid flow circulates through the tissue modeling environment by travelling through the first microchannel 125a. The first fluid reservoir 110a and the third fluid reservoir 110c are fluidically coupled by the first microchannel 125a defined in the underside of the second structural layer 560. When the first fluid reservoir 110a and the third fluid reservoir 110c each have a column of fluid of equal height, there is no gravity fed fluid flow through the first microchannel 125a. In order to create a difference in height between the columns of fluid in the first fluid reservoir 110a and the third fluid reservoir 110c, the pump assembly 115 pumps a first fluid 180 out of the second fluid output valve 155b into the third fluid reservoir 110c. Introducing the first fluid 180 into the third fluid reservoir 110c creates a difference in height between the columns of fluid in the first fluid reservoir 110a and the third fluid reservoir 110c causing a gravity fed fluid flow 180 from the first fluid reservoir 110c to the second structural layer 560. The fluid flow 180 enters the second structural layer 560 through a bore hole 585. The first fluid flow 180 travels across the first microchannel 125a defined in the second structural layer 560. The fluid flow 180 exits the first microchannel 125a and the second structural layer 560 via another bore hole 585. The first fluid flow 180 enters the first fluid reservoir 110a causing the fluid column height between the first fluid reservoir 110a and the third fluid reservoir 110c to equalize. Once the fluid column height between the first fluid reservoir 110a and the third fluid reservoir 110c equalizes, there will no longer be a gravity fed fluid flow through the first microchannel 125a. In order to maintain the gravity fed fluid flow 180 through first microchannel 125a, a difference in fluid column height between the first fluid reservoir 110a and the third fluid reservoir 110c needs to be maintained. Therefore, the pump assembly 115 pumps the first fluid 180 out of the first fluid reservoir 110a through the first fluid input valve 150a repeatedly creating a difference in fluid column height between the first fluid reservoir 110a and the third fluid reservoir 110c.

In FIG. 5B, a second gravity fed fluid flow circulates through the tissue modeling environment by travelling through the second microchannel 125b. The second fluid reservoir 110b and the fourth fluid reservoir 110d are fluidically coupled by the second microchannel 125 b defined in the underside of the second structural layer 570. When the second fluid reservoir 110b and the fourth fluid reservoir 110d each have a column of fluid of equal height, there is no gravity fed fluid flow through the second microchannel 125b. In order to create a difference in height between the columns of fluid in the second fluid reservoir 110b and the fourth fluid reservoir 110d, the pump assembly 115 pumps a second fluid 185 out of the second fluid input valve 150b into the fourth fluid reservoir 110d. Introducing the second fluid 185 into the fourth fluid reservoir 110d creates a difference in height between the columns of fluid in the second fluid reservoir 110b and the fourth fluid reservoir 110d and causes the gravity fed second fluid flow 185 to travel from the second fluid reservoir 110b into the second structural layer 560. The second fluid flow 185 enters and exits the second structural layer 560 through a bore hole 585. The second fluid flow 185 enters and exits the membrane 140 through another bore hole 585 and travels across the second microchannel 125b defined in the third structural layer 570. The second fluid 185 exits the second microchannel 125b and travels through the second structural layer 560 via the bore hole 585. The first fluid flow 180 enters the second fluid reservoir 110b causing the fluid column height between the second fluid reservoir 110b and the fourth fluid reservoir 110d to equalize. Once the fluid column height between the second fluid reservoir 110b and the fourth fluid reservoir 110d equalizes, there will no longer be gravity fed fluid flow through the second microchannel 125b. In order to maintain the gravity fed fluid flow 185 through second microchannel 125b, a difference fluid column height between the second fluid reservoir 110b and the fourth fluid reservoir 110d needs to be maintained. Therefore, the pump assembly 115 pumps the second fluid 185 out of the second fluid reservoir 110b through the second fluid input valve 150b once again creating a difference in fluid column height between the second fluid reservoir 110b and the fourth fluid reservoir 110d. In some implementations, the first fluid 180 and the second fluid 185 recirculate within one tissue modeling environment. In some implementations, the first fluid 180 and the second fluid 185 are not recirculated but are rather moved out of the tissue modeling environment by the pump assembly 115. In other implementations, the first fluid 180 and the second fluid 185 are moved between different tissue modeling environments. In other implementations, the first fluid 180 and the second fluid 185 are introduced to the fluid reservoirs 110c and 110d respectively from a fluid source outside the tissue modeling environment rather than being introduced from the fluid reservoirs 110a and 110b. In some implementations, the first fluid 180 and the second fluid 185 are drained to a fluid sink that resides outside the tissue modeling environment. In some implementations, the fluid source and the fluid sink may be one or more tissue modeling environments. In some implementations, multiple tissue modeling environments in the cell culture platform 105 may be interconnected. The flow rate with a tissue modeling environment may be changed by changing the pump rate, the input valve or output valve sipper depth, the pump chamber diaphragm size or the bore height. In some implementations, fluid flow through the first microchannel 125a above the membrane 140 and fluid flow through the second microchannel 125b below the membrane 140 can each produced by dedicated pump chambers, input and output valves, and fluid reservoirs to perfuse the first microchannel 125a and the second microchannel 125b independently of each other. In some implementations, the first microchannel 125a or the second microchannel 125b or both microchannels 125a and 125b may have a fluid flow rate of zero.

In some implementations, the cell culture platform 105 includes components configured to enable a biochemical reading via optical sensors, electrode traces or other biocompatible sensors. In some implementations, the sensors may be connected to external sensing hardware, which in turn may be coupled to the controller mentioned above. In some implementations, the sensors provide real-time and direct quantification of cell culture conditions and tissue response. Parameters such as tissue culture health, quality, morphology, confluence etc. can be monitored and evaluated without having to remove the cell culture platform 105 from an incubator. The optical sensors may have a fluorescence or phosphorescence that is modulated by the concentration of molecules of glucose, oxygen, or other analytes. The electrode trace may include silver chloride, gold, platinum or other biocompatible conductors. In some implementations, the electrodes are configured to stimulate and record electrical signals to, for example, generate a TransEpithelial Electrical Resistance (TEER) profile. TEER is used, in some implementations, to measure the integrity and health of the tissues cultured in the tissue modeling environment.

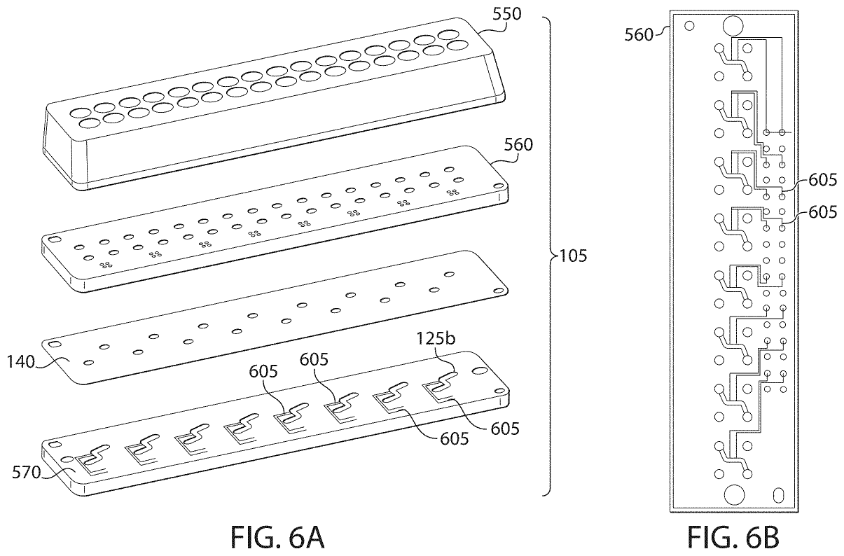

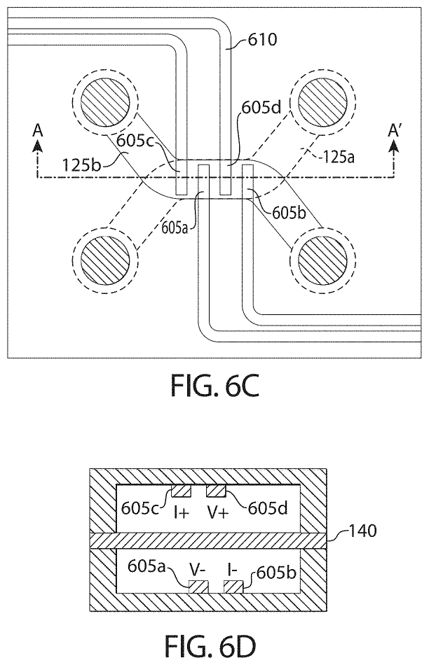

FIG. 6A illustrates an exploded view of an example cell culture platform 105 having an array of tissue modeling environments with integrated sensors. FIG. 6A, includes a first structural layer 550, a second structural layer 560, a membrane 140, and a third structural layer 570. The second structural layer 560 defines a first set of microchannel such as the first microchannel 125a. The third structural layer 570 defines a second set of microchannel structures, such as a second microchannel 125b. The third structural layer 570 includes an array of electrodes 605 with each microchannel such as the second microchannel 125b having two dedicated electrodes 605. In FIG. 6A, the array of electrodes 605 are printed onto the second set of microchannels 125b to make a continuous and conformal electric connection between the electrodes 605 in the microchannels 125b and external sensing hardware. FIG. 6B illustrates a top side down view of the second structural layer 560 of the example cell culture platform 105 illustrated in FIG. 6A. In some implementations, the electrode 605 may be electrode traces having a line width of about 100 microns.

FIG. 6C illustrates a top view of an example cell culture environment with integrated sensors. FIG. 6C is a top viewing looking down through the second structural layer and the third structural layer. In FIG. 6C, the cell culture platform includes a first microchannel 125a, a second microchannel 125b, a plurality of traces 610, and a plurality of electrodes 605. The cell culture environment includes an array of electrodes 605, such as a first electrode 605a, a second electrode 605b, a third electrode 605c, and a fourth electrode 605d. The electrodes 605 are routed to traces 610. The traces apply current and voltage to the electrodes 605 from current sources and voltage sources located outside the cell culture platform. The first electrode 605a and the second electrode 605b are printed onto the second microchannel 125b and the third electrode 605c and the fourth electrode 605d are printed on the first microchannel 125a.

FIG. 6D illustrates a cross sectional view the cell culture platform shown by the line labeled A-A′ in FIG. 6C. FIG. 6D includes a first microchannel 125a, a second microchannel 125b, a membrane 140, and a plurality of electrodes 605. The membrane separates the first microchannel and the second microchannel 125b. The first microchannel includes a third electrode 605c and a fourth electrode 605d and the second microchannel includes a first electrode 605a and a second electrode 605b.

In some implementations, the first microchannel 125a and the second microchannel 125b each include a pair of electrodes. In some implementations, only one of the first microchannel 125a and the second microchannel 125b includes electrodes. In some implementations, a single pair of electrodes is placed across the membrane 140 (i.e., one in each microchannel) may measure the electrochemical impedance of a cell layer across the membrane using electrochemical impedance spectroscopy to measure electrical resistance over a range of frequencies. In some implementations, a set of four electrodes may be used (i.e., two in each microchannel) as a four point probe to measure the response and impedance at a single frequency. In some implementations, a pair of electrodes may be placed in the first or second microchannel 125a or 125b and only one electrode may be place in the other microchannel and still serve as a four-point probe. In some implementations, the microchannel structure of a tissue modeling environment may be fabricated by embossing a plastic material.

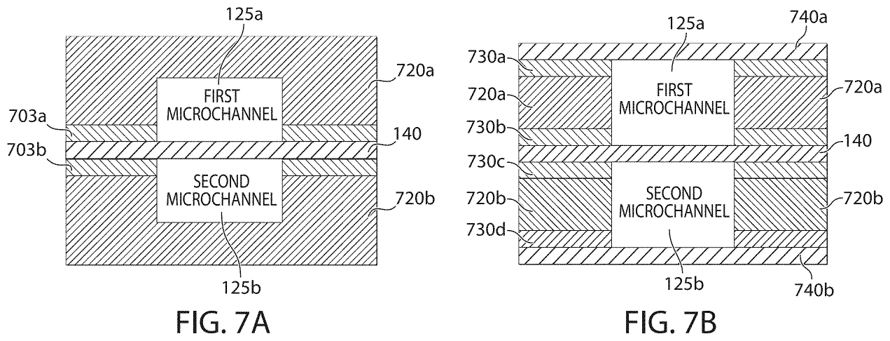

FIG. 7A illustrates a cross section of a microchannel structure fabricated by embossing a plastic material. In FIG. 7A, the microchannels 125 include a first piece of embossed plastic material 720a and a second piece of embossed plastic material 720b, a membrane 140, a first layer of adhesive film 730a and a second layer of adhesive film 730b. The first and second pieces of embossed plastic material 720a and 720b are each a plastic material embossed with a cavity in the form of a microchannel. The fabrication process includes attaching the first layer of adhesive film 730a to the first piece of embossed plastic material 720a and attaching the second layer of adhesive film 730b to the second piece of embossed plastic material 720b. Portions of the first and second layers of adhesive film 730a and 730b are cut out or removed around and inside the microchannels 125 and ports allowing fluid to flow through the ports and into the microchannels 125. The first and second layers of adhesive film 730a and 730b are attached to the membrane 140. In some implementations, the various components may be attached using thermal or pressure sensitive adhesives. In some implementations, the membrane 140 may manufactured from be a track-etched polycarbonate membrane. In some implementations, the first and second embossed plastic material 720a and 720b and the adhesive film 730 may be manufactured from cyclic olefin co-polymer (COC) or gas/oxygen permeable polymers such as fluorinated ethylene propylene (FEP) or polymethylpentene (PMP), polyurethane, polystyrene or polysulfone. In some implementations of the fabrication process, portions of the membrane 140 may be plasma activated or exposed to UV through a photolithographic mask to create free radical and promoting the cells to adhere to the membrane 140. In some implementations in which the microchannel structure in a tissue modeling environment is fabricated using an embossed plastic material, a plurality of electrode sensors may be printed onto the various layers using 3-D printing techniques.

In some implementations, the microchannel structure of a tissue modeling environment may be fabricated using a thru-cut technique. FIG. 7B illustrates a cross section of a microchannel structure fabricated using a thru cut technique. In FIG. 7B, the microchannel structure includes a first layer of plastic material 720a, a second layer of plastic material 720b, a membrane 140, a first layer of adhesive film 730a, a second layer of adhesive film 730b, a third layer of adhesive film 730c, a fourth layer of adhesive film 730d, a first layer of thin film 740a and a second layer of thin film 740b. The fabrication process includes attaching the first layer of adhesive film 730a to a first side of the first plastic material 720a and attaching the second layer of adhesive film 730b to a second side of the first plastic material 720a. The fabrication process further includes attaching the third layer of adhesive film 730c to a first side of the second layer of plastic material 720b and attaching the fourth layer of adhesive film 730d to a second side of the second plastic material 720b. Portions of the first, second, third and fourth layers of adhesive film 730a-730d and portions of the first and second layers of plastic material 720a and 720b are cut out or removed from areas around and inside the microchannels 125 and ports allowing fluid to flow through the ports and into the microchannels 125. A first side of the membrane 140 is attached to portions of the second layer of adhesive film 730b and a second side of the membrane 140 is attached to portions of the third layer of adhesive film 730c. The microchannel structure is stabilized by adhering a first layer of thin film 740a to the first layer of adhesive film 730a and adhering a second layer of thin film 740a to the fourth layer of adhesive film 730d.

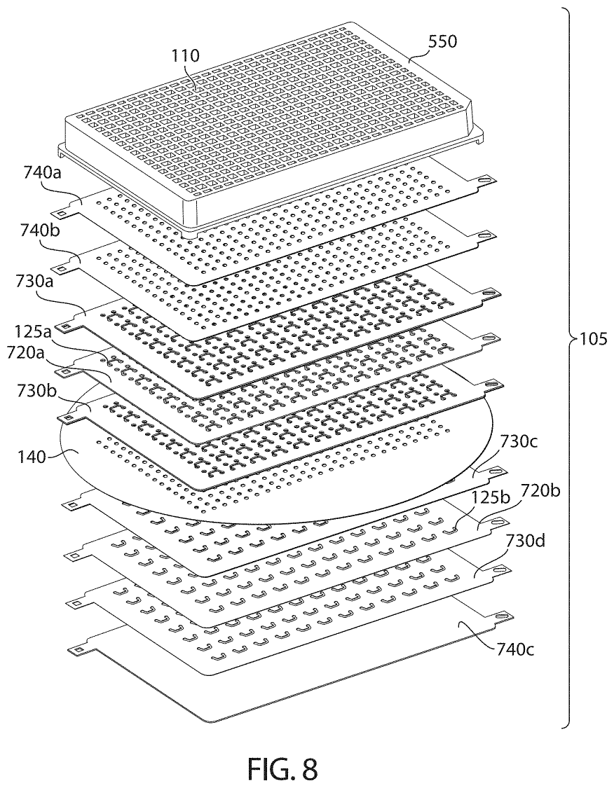

FIG. 8 illustrates an exploded view of an example cell culture platform fabricated using a thru-cut technique, as previously shown in FIG. 7B. An array of tissue modeling environments in a cell culture platform is defined by several structural layers separated by a membrane 140. In FIG. 8 the microchannel structure includes a first layer of plastic material 720a and a second layer of plastic material 720b, a first layer of adhesive film 730a, a second layer of adhesive film 730b, a third layer of adhesive film 730c, and a fourth layer of adhesive film 730d, a first layer of thin film 740a, a second layer of thin film 740b, and a third layer of thin film 740c. FIG. 8 also includes a membrane 140, microchannel structures 125a and 125b, a first structural layer 550, and a plurality of fluid reservoirs 110. The first microchannel 125a is formed by a cut through in the first layer of adhesive film 730a, the first layer of plastic material 720a, and the second layer of adhesive film 730b. The second microchannel 125 is formed by a cut through in the third layer of adhesive film 730c, the first layer of plastic material 720b, and forth layer adhesive film 730d.

The fabrication of the cell culture platform includes attaching the first structural layer 550 to the first layer of thin film 740a. It further includes attaching the second layer of thin film 740 b to the second side of the first layer of thin film 740a and to the first side of the first layer of adhesive film 730a. Fabrication further includes attaching the second side of the first layer of adhesive film 730a to the first layer of plastic material 720a. Fabrication further includes attaching the second side of the first plastic material 720a to the second layer of adhesive film 730b. The first side of the membrane 140 attaches to portions of the second layer of adhesive film 730b and to portions of the third layer of adhesive film 730c. The third layer of adhesive film 730d attaches to the second layer of plastic material 720b and the second side of the second layer of plastic material attaches to the fourth layer of adhesive film 730d. The second side of the fourth layer of adhesive film 730d attaches to the third layer of thin film 740c. The first layer of thin film 740a, the second layer of thin film 740b, and the third layer of thin film 740c provide stabilization to the microchannel structure 125. In some implementations, the adhesive film may be manufactured from an epoxy or curable adhesive. In some implementations, the adhesive film may be manufactured from an adhesive tape such as a pressure sensitive adhesive. In some implementations, the adhesive film may be manufactured from a material that has been fluoroetched, plasma treated, chemically etched, or surface patterned to enhance its adhesive properties. In some implementations, the adhesive film may be the same class of polymer as the plastic material but with a different melting point or glass transition temperature. In some implementations, the adhesive film may be about 1.0 to 25 μm thick.

In some implementations, the various components may be attached using thermal or pressure sensitive adhesives. In some implementations, the membrane 140 may be manufactured from a track-etched polycarbonate membrane. In some implementations, the first and second layer of plastic material 720a and 720b, the first, second, third and fourth layer of adhesive film 730a-730d and the first, second, and third layer of thin film 740a, 740b, and 740c, respectively, may be manufactured from COC or gas/oxygen permeable polymers such as fluorinated ethylene propylene (FEP) or polymethylpentene (PMP), polyurethane, polystyrene or polysulfone. In some implementations, the third layer of thin film 740c may be manufactured from an oxygen permeable material such as FEP which may allow fluid flow to be decoupled from oxygen requirements, or enable static cell cultures where there is no flow in either microchannel. In some implementations, the third layer of thin film 740c may be manufactured from an oxygen impermeable material in order to control the oxygen environment through the fluid flow or lack thereof and create hypoxic conditions. In some implementations in which the microchannel structure in a tissue modeling environment is fabricated using a thru cut technique, a plurality of electrode sensors may be formed using lithography.

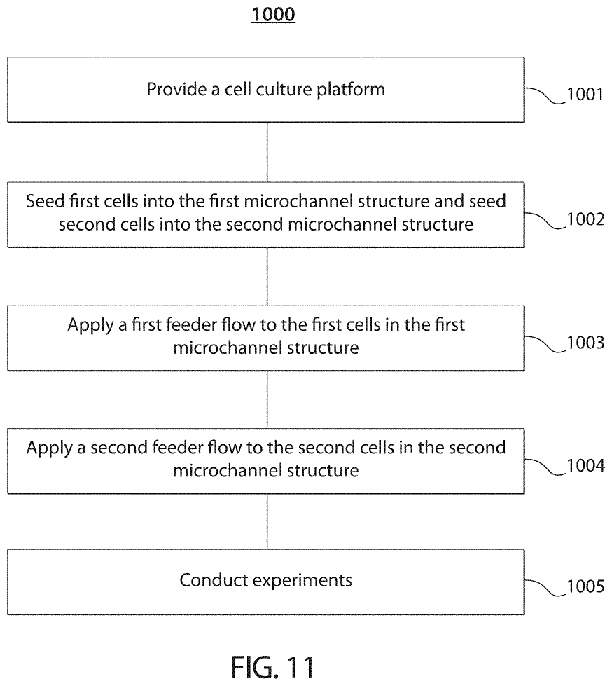

Referring to FIGS. 1-10, FIG. 11 illustrates a flow chart of an example method 1000 for populating cells into the cell culture platform. The method 1000 includes providing a cell culture platform and a plurality of cells (step 1001). Then, a first cell type is seeded into a first microchannel structure and a second cell type is seeded into a second microchannel structure (step 1002). Next, the method 1000 includes applying a first feeder flow to the first cells (step 1003). Then, applying a second feeder flow to the second cells (step 1004). Experiments may be conducted across the populated cells in the cell culture platform (step 1005).

As set forth above, the method 1000 begins with the provision of a cell culture platform including a plurality of tissue modeling environments and a plurality of cells (step 1001). In some implementations, the tissue modeling environments may be similar to the tissue modeling environments described in FIG. 1-10 above. For example, the tissue modeling environment includes a group of fluid reservoirs 110 fluidically coupled by a pair of microchannel structures 125, including a first microchannel 125a and a second microchannel 125b separated by a membrane 140, as shown in FIG. 3A and FIG. 3B. The tissue modeling environments can be arrayed in a cell culture platform such as the platform 105 shown in FIG. 1.

A first cell type is seeded into the first microchannel 125a while a second cell type is seeded into the second microchannel 125b (step 1002). In some implementations, the first microchannel structure 125b represents an apical channel and the second microchannel structure represents a basal channel. In some implementations, the first cell type may be epithelial cells and the second cell type may be microvascular cells. In other implementations, the cell culture platform may approximate the in vivo structure of a renal tubule, where the first cell type may be renal proximal epithelial cells and the second cell type may be endothelial cells. The cells can be seeded into the respective channels by disposing the cells into reservoirs of the respective cell tissue culture environments, and allowing the cells to flow through the microchannels and the pump assembly as fluid is extracted from outlet reservoirs and reintroduced into inlet reservoirs of the tissue culture environments until the cells adhere to the membrane in the microchannels. A first feeder flow is applied to the first cell type in the first microchannel 125a (step 1003). In some implementations, the feeder flow is applied to the cells at a rate of about 1 μL/min. In other implementations, the feeder flow is applied to the first cells at a rate less than 1 μL/min. The first feeder flow can include cell culture media typically used for culturing cells. In some implementations, the first feeder flow can include a proliferative cell culture medium. In some implementations, the first feeder flow can include several components or supplements of cell culture medias, mixed to create an environment conducive for growth, differentiation, or survival of multiple cell types. In some implementations, the first feeder flow may be a buffer or saline solution.

A second feeder flow is applied to the second cell type in the second microchannel 125b (step 1004). In some implementations, the second feeder flow is applied to the second cell type about 24 hours after the first feeder flow was applied to the first cell type. In some implementations, feeder flow is applied to the second cell type at a rate of about 1 μL/min. The second feeder flow can include cell culture media typically used for culturing cells. In some implementations, the second feeder flow can include can include a proliferative cell culture medium. In some implementations, the second feeder flow can include several components or supplements of cell culture medias, mixed to create an environment conducive for growth, differentiation, or survival of multiple cell types. In some implementations, the second feeder flow may be a buffer or saline solution. In some implementations, the fluid flow can be used to condition cells, maintain cell growth, differentiate cells, profuse the tissue, seed cells, and/or administer mechanical stresses and forces.

The first microchannel 125a can have a first fluid flow and the second microchannel 125b can have a second fluid flow. In some implementations, the first microchannel 125a and the second microchannel 125b can have the same flow rate. In some implementations, after the cells have been cultured in their respective microchannels 125a and 125b for an initial amount of time (e.g., about 24-48 hours), the pump rate may increase to about 1 μL/sec in both the first microchannel 125a and the second 125b structure to mimic physiological shear stress on the cells. In some implementations, the first flow rate or the second flow rate may increase to a rate which exerts about 0.1 Pa of pressure across the cell membrane 140, thereby mimicking a kidney proximal tubule.

The method 1000 further includes conducting experiments upon the cells in the culture platform. In some implementations, the experiment may measure barrier function across the cell membrane over the course of several days. In some implementations, the experiment may calculate the rate of transport across the membrane 140. In some implementations, the cell culture platform mimics an organ system by introducing a plurality of molecules to a specific cell type on the membrane 140 and the experiment measures transport. For example, a user may combine a liquid-gas molecule into a cell culture platform configured with alveolar cells on the membrane to measure real-time transport in the lungs. In some implementations, a user may couple multiple tissue modeling environments with different cell types to mimic a plurality of organ systems. In some implementations, the plurality of molecules may represent a specific drug and the experiment provides a drug to tissue delivery analysis. In some other implementations, biologically active agents, such as a drugs, toxins, chemotherapeutics, nutrients, bacteria, viral particles, etc., are pumped through the cell culture environments at the same or different concentrations and/or flow rates to measure the impact of such agents on the cell culture environments.

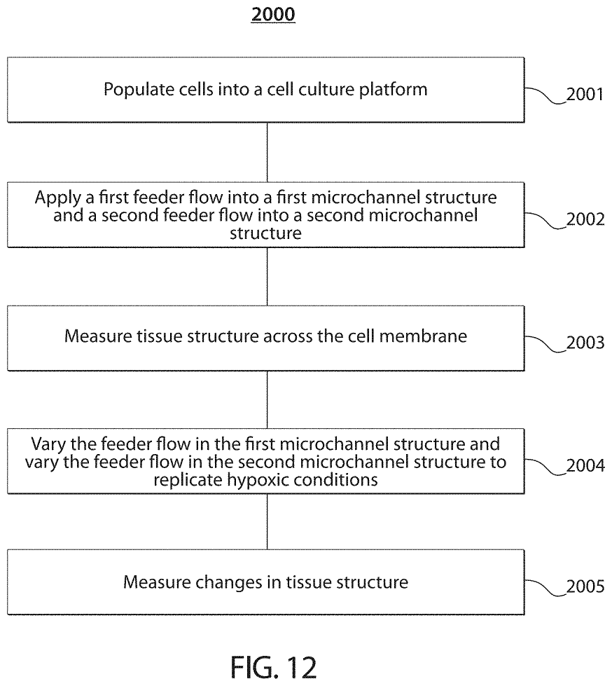

Referring to FIGS. 1-10, FIG. 12 illustrates a flow chart of an example experimental method 2000 for simulating hypoxic conditions in healthy tissue. In some implementations, the cell culture platform controls oxygen levels in one or more channels to mimic hypoxic conditions in healthy tissue, such as the gut microenvironment, disease states, or ischemia in the kidneys. The method 2000 includes populating a cell culture platform with a plurality of cells (step 2002), as shown in example method 1000 above. Then, the method 2000 includes applying a feeder flow into a first microchannel structure and a feeder flow into a second microchannel structure (step 2002). Method 2000 includes measuring the tissue structure across the cell membrane (step 2003). The feeder flow is varied in the first microchannel structure and the second microchannel structure to replicate hypoxic conditions (step 2004). The method 2000 further includes measuring changes in the tissue structure across the cell membrane (step 2005) due to the replicated hypoxic condition.

As set forth above, the method 2000 begins with populating a cell culture platform with a plurality of cells (step 2001), similar to example method 1000 above. In some implementations, the cell culture platform can be similar to cell culture platforms 105 described in FIG. 1-10. For example, the cell culture platform may include a first microchannel 125a, a second microchannel 125b, a membrane 140 separating the first microchannel 125a and the second microchannel 125b, and a group of fluid reservoirs 110 fluidically coupled by the first microchannel structure 125a and the second microchannel structure 125b, as shown in FIG. 3A and FIG. 3B. Multiple cell culture environments can be arrayed across the cell culture platform, e.g., with the reservoirs of the cell culture environments having an arrangement similar to a standard well plate arrangement. In some implementations, the first and the second microchannel structures may be formed in a gas-impermeable polymer.

Next, the method 2000 includes applying a first feeder flow into a first microchannel 125a and a second feeder flow into a second microchannel 125a (step 2002). In some implementations, the pump assembly 115, similar to FIG. 5A, applies the feeder flow to the cells at a rate greater than 1 μL/min to keep the oxygen levels of the cells high. Then, method 2000 includes measuring the tissue structure across the cell membrane (step 2003). For example, TEER measurements can be made across the cells coupled to the cell membrane.

Next, the method 2000 includes varying the feeder flow in the first microchannel 125a as well as varying the feeder flow in the second microchannel 125b to replicate a hypoxic condition (step 2004). In some implementations, the pump assembly 115 varies the flow rate to less than in order to lower the oxygen levels in the cells. In other implementations, low oxygen content fluid can be delivered at a suitable flow rate. In other implementations, the top of the fluid reservoirs 110 may be blocked to limit the introduction of environmental oxygen into the fluid flows and thus to the cells. Next, step 2005 includes measuring changes in the tissue structure or, e.g., the TEER response of the cells, in response to the varying oxygen levels.

### EXAMPLES

The function and advantages of these and other embodiments can be better understood from the following examples. These examples are intended to be illustrative in nature and are not considered to be limiting the scope of the invention.

### Example 1: Prophetic Example of Measuring Oxygen Consumption Rate of Cells

The microfluidic device of the exemplary embodiment may contain two microchannels with a porous, thin, polymer membrane in between the two channels. The two channels may form an upper and lower channel. An oxygen sensor may be deposited within the lower microchannel, along the bottom surface (below the membrane) approximately 200 μm from the membrane. The deposited sensor may be an optical based sensor. The bottom layer may be optically clear to allow for interfacing between the deposited sensor and the optical based oxygen sensing hardware (fiber optic cable and/or microscope). The oxygen sensing hardware may be situated external to the microfluidic device.

One or more cell types may be cultured on the top and/or bottom surface of the membrane. Pumps may be positioned in the inlet and/or outlet wells that interface the top and bottom microchannels. The pumps may be configured to provide fluid flow from the inlet wells, through both microchannels to the outlet wells, and then back through the pumps to recirculate flow to the inlets.

The method may enable sensitive and non-contact measurement of cell oxygen consumption by entrapping the cells within a small liquid volume surrounded by oxygen impermeable polymer and an oxygen sensor, control of oxygen supply to the microchannel via the pumps, and positioning the cells on a membrane or scaffold to keep cells from contacting the sensor.

To measure oxygen consumption rate, the cells are cultured in the microchannel cell culture device. In particular, the cells may be cultured along the central porous membrane. One or more cell types of interest may be cultured.

Pumps may be activated to deliver oxygen saturated fluid, generating continuous and high oxygen levels within the device. Activation of the pumps may result in transport of highly oxygenated fluid from the inlet wells through the channel and establishes steady high oxygen levels within the microchannels. Oxygen levels may be measured under continuous flow.

Pumps may be turned off to cut off oxygen supply to the microchannels. Deactivation of the pumps may result in a halt to transport of oxygenated fluid from the inlet wells to the channels. With pumps turned off, it is expected that there would be no significant oxygen transfer into the microchannel from the wells. Oxygen transfer through the walls of the channel is expected to be insignificant due to low oxygen permeability of the channel layers. Oxygen levels may be measured under no flow conditions.

The subsequent oxygen depletion due to cell consumption may be measured to determine the oxygen consumption rate of the cells. Flow may be re-initiated to repeat the measurement process.

### Example 2: Microchannel Cell Culture System Set-Up and Oxygen Consumption Rate Measurements

Objective

The objective of the study was to provide a system and method for optical-based label-free and non-invasive measurement of cell oxygen consumption, a key parameter for assessing tissue metabolic function, within a multi-well microfluidic tissue culture system. Additionally, an aim of this study was to measure drug effects on tissue metabolic function within a microfluidic tissue culture system for drug development applications. However, the optical-based measurement approach developed herein may be effectively applied for measuring other biologically significant changes within the tissue culture environment, such as changes in acidification, glucose, or secreted molecules.

Experimental Setup

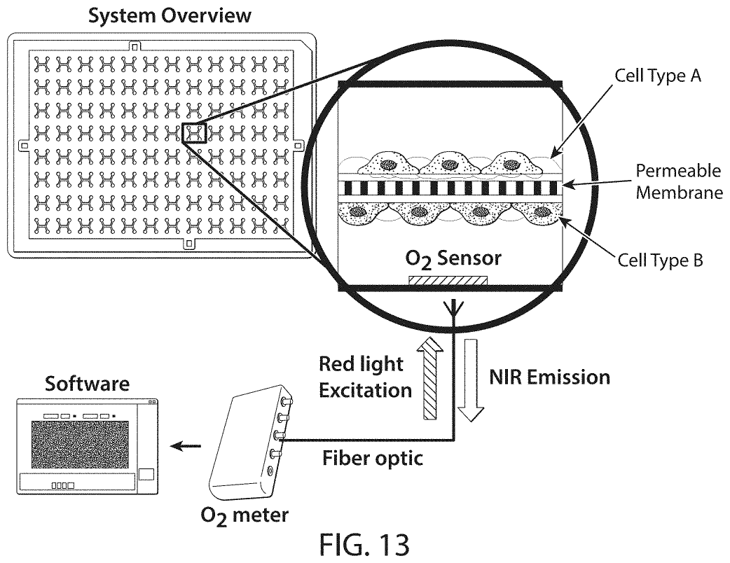

An optical luminescence-based oxygen sensing system was integrated with an existing multi-well microfluidic tissue culture device, for label-free oxygen monitoring (FIG. 13). Sensor spots having a diameter of 0.75 mm were cut with a biopsy punch from a sheet of photosensitive film (PyroScience) and bonded with 184 Sylgard silicone adhesive to the center floor of each basal channel. The photosensitive sensor spot was located about 200 μm from the tissue layer on the surface of bottom channel. A FireStingO₂ optical oxygen meter (PyroScience) with a 2 m fiber optic cable (external to channel) was used to monitor oxygen in each device.

For oxygen measurements, the device was placed in an incubated confocal microscope (Zeiss Inc., Oberkochen, Germany) and the fiber optic was secured below the microscope stage. The stage was programmed to align the fiber optic with each sensor for oxygen monitoring in all 96 tissue modeling environments. A 2-point calibration of the FireStingO₂ system was performed using devices filled with air saturated PBS and a 0% oxygen solution (30 g/L sodium sulfite in water). Human renal proximal tubule cells were cultured along the device's central permeable membrane.

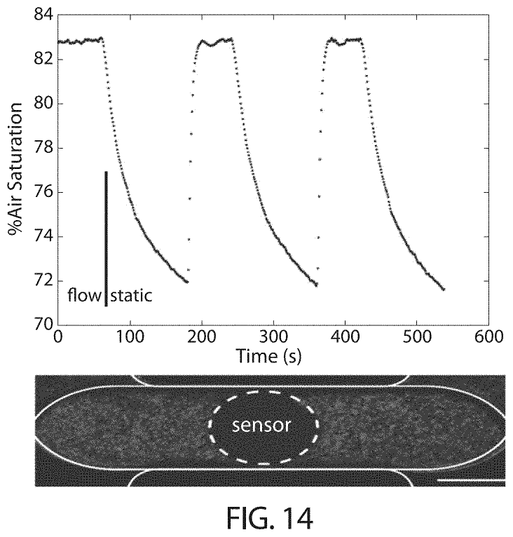

Tissue oxygen consumption was measured for the human renal proximal tubule cells by cycling the pumps carrying oxygen rich fluid on and off (FIG. 14). As shown in the graph of FIG. 14, under static conditions, cell oxygen consumption was measured immediately upon turning off fluid pumps. The location of the sensor in the bottom center of the microchannel is shown in FIG. 14.

Results

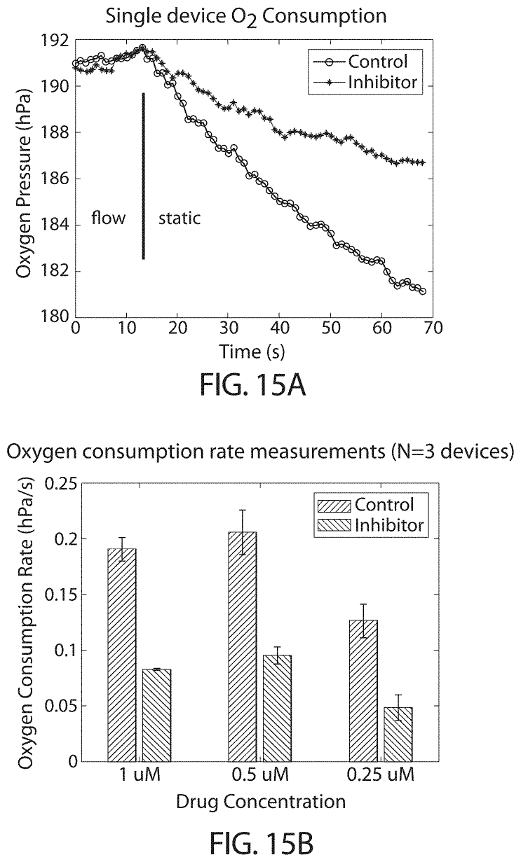

Under flow, oxygen measurements remained steady at high oxygen concentrations. Under static conditions, oxygen measurements declined immediately, reflecting cell oxygen consumption within each device. FIG. 15A shows the measurement of drug-induced shifts in cell oxygen consumption (measured as oxygen pressure). As shown in the data presented in FIG. 15B, a significant decrease in cell oxygen consumption rate was measured for human renal proximal tubule cells treated with Oligomycin, a known drug that inhibits cell mitochondrial respiration.

### DISCUSSION

The described method for integration of an optical based sensing system with an existing microchannel cell culture device and measurement of oxygen consumption demonstrates a valuable approach for label-free tissue metabolic sensing within microfluidic tissue culture platforms.

Sensor integration with microfluidic systems allows high-throughput oxygen readouts for 96 microfluidic tissue culture environments on a single plate. Activation and deactivation of the fluid pumps allows for sensitive oxygen consumption measurements of tissue cultured within each environment. Additionally, the described method has demonstrated the capability for measurement of drug-induced shifts in cell oxygen consumption, which provides a useful tool for assessing drug effects on tissue metabolism during pre-clinical drug development.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. A cell culture system comprising:
a microchannel cell culture device, comprising
a well plate formed of a plurality of structural layers and a membrane layer, the membrane layer positioned between two structural layers and the well plate defining an array of tissue modeling environments, each tissue modeling environment including:
a first fluid reservoir, a second fluid reservoir, a third fluid reservoir, and a fourth fluid reservoir;
a first microchannel fluidically coupling the first fluid reservoir to the second fluid reservoir; and
a second microchannel fluidically coupling the third fluid reservoir to the fourth fluid reservoir, at least a portion of the first microchannel overlapping at least a portion of the second microchannel, the membrane layer extending between the overlapping portions of the first and second microchannel, each of the overlapping portions of the first and second microchannels being optically transparent;
a plurality of optical sensors, each optical sensor positioned to scan a corresponding overlapping portion of the first and second microchannels on a bottom surface of the well plate; and
a platform configured to support a bottom surface of the microchannel cell culture device.

2. The system of claim 1, further comprising a light source configured to be positioned adjacent the platform opposite the microchannel cell culture device, the light source configured to direct light towards the plurality of optical sensors; and
a meter operably connected to the light source.

3. The system of claim 1, wherein the optical sensors are formed of a nanoparticle solution.

4. The system of claim 3, wherein the light source is a fiber optic cable.

5. The system of claim 4, wherein the platform is movable.

6. The system of claim 1, wherein the optical sensor is configured to measure at least one of oxygen concentration, pH, temperature, and glucose concentration.

7. The system of claim 1, wherein the well plate comprises a light shielding layer positioned on a top surface of the well plate.

8. The system of claim 1, wherein each optical sensor has a length extending from the first fluid reservoir to the second fluid reservoir, and from the third fluid reservoir to the fourth fluid reservoir.

9. The system of claim 1, wherein each fluid reservoir is configured to hold a column of fluid.

10. A microchannel cell culture device, comprising:
a well plate defining an array of tissue modeling environments, each tissue modeling environment including at least one microchannel fluidically coupling a first fluid reservoir to a second fluid reservoir, a bottom surface of the at least one microchannel being optically transparent;
a light shielding layer positioned adjacent a top surface of the at least one microchannel; and
a plurality of optical sensors, each optical sensor positioned to scan the bottom surface of the at least one microchannel of a corresponding tissue modeling environment.

11. The microchannel cell culture device of claim 10, wherein each tissue modeling environment includes at least two microchannels, a first microchannel fluidically coupling the first fluid reservoir to the second fluid reservoir and a second microchannel fluidically coupling a third fluid reservoir to a fourth fluid reservoir.

12. The microchannel cell culture device of claim 11, wherein at least a portion of the first microchannel overlaps at least a portion of the second microchannel.

13. The microchannel cell culture device of claim 12, further comprising a membrane layer extending between the overlapping portions of the first and second microchannels.

14. The microchannel cell culture device of claim 13, wherein a bottom surface of the overlapping portions of the first and second microchannels is optically transparent.

15. A method of high throughput screening cell biological activity, comprising:
seeding at least one cell type onto at least one tissue modeling environment of a microchannel cell culture device comprising:
a well plate formed of a plurality of structural layers and a membrane layer, the membrane layer positioned between two structural layers and the well plate defining an array of the tissue modeling environments, each tissue modeling environment including:
a first fluid reservoir and a second fluid reservoir; and
a microchannel fluidically coupling the first fluid reservoir to the second fluid reservoir;
a bottom surface of the microchannel being optically transparent; and
a plurality of optical sensors, each optical sensor positioned to scan the bottom surface of the microchannel of a corresponding tissue modeling environment;
introducing a pre-determined dose of at least one biologically active agent into the at least one tissue modeling environment; and
measuring a parameter within the at least one tissue modeling environment to produce a first measurement.

16. The method of claim 15, further comprising:
positioning the microchannel cell culture device on a platform configured to support the bottom surface of the microchannel cell culture device; and
activating a light source positioned adjacent the platform opposite the microchannel cell culture device to direct light towards the plurality of optical sensors.

17. The method of claim 16, further comprising:
after a pre-determined amount of time, taking a second measurement of the parameter within the at least one tissue modeling environment; and
calculating a rate of change of the parameter from the first and second measurement to determine the cell biological activity of the at least one cell type responsive to the at least one biologically active agent.

18. The method of claim 15, further comprising coupling the light source to a surface of the platform opposite the microchannel cell culture device.

19. The method of claim 15, wherein the parameter is selected from oxygen concentration, pH, temperature, and glucose concentration.

20. A method of measuring oxygen consumption rate of cells, comprising:
seeding the cells onto at least one tissue modeling environment of the microchannel cell culture device of claim 10;
introducing an oxygen rich fluid into the at least one seeded tissue modeling environment;
measuring a first oxygen concentration within the at least one seeded tissue modeling environment with the plurality of optical sensors;
reducing flow rate of the oxygen rich fluid to induce a static environment within the at least one seeded tissue modeling environment; and
after a pre-determined amount of time, measuring a second oxygen concentration within the at least one seeded tissue modeling environment with the plurality of optical sensors to determine the oxygen consumption rate of the cells.

21. A method of facilitating drug development, comprising:
providing the cell culture system of claim 1; and
providing instructions to:
seed at least one cell type onto at least one tissue modeling environment;
introduce a pre-determined dose of at least one biologically active agent of the drug into the at least one tissue modeling environment; and
measure a parameter within the at least one tissue modeling environment to produce a first measurement.

22. The method of claim 21, further comprising providing instructions to:
activate a light source to direct light towards the plurality of optical sensors.

23. The method of claim 22, further comprising, providing instructions to:
after a pre-determined amount of time, measure the parameter within the at least one tissue modeling environment to produce a second measurement; and
calculate a rate of change of the parameter from the first and second measurement to determine the cell biological activity of the at least one cell type responsive to the at least one biologically active agent.

24. The method of claim 21, further comprising providing a platform configured to support the bottom surface of the microchannel cell culture device.

25. The method of claim 24, further comprising providing a light source configured to be positioned adjacent the platform opposite the microchannel cell culture device, the light source configured to direct light towards the plurality of optical sensors.
